(19) United States
(12) Patent Application Publication
(10) Pub. No.: US 2025/0236810 A1
(43) Pub. Date: Jul. 24, 2025

(54) FRAGRANCE AND FLAVOR COMPOSITIONS COMPRISING BENZENE DERIVATIVES

(71) Applicant: OSMO LABS, PBC, Cambridge, MA (US)

(72) Inventors: Alexander WILTSCHKO, Somerville, MA (US); Christophe LAUDAMIEL, New York, NY (US); Bradley B. GILBERT, Milford, NJ (US); Andrew P. PATRON, Oceanside, CA (US); Benjamin AMORELLI, Brielle, NJ (US)

(21) Appl. No.: 19/036,841

(22) Filed: Jan. 24, 2025

Related U.S. Application Data
(60) Provisional application No. 63/624,743, filed on Jan. 24, 2024.

(51) Int. Cl. C11B 9/00; C07C 43/205; C07C 69/76

(52) U.S. Cl. CPC C11B 9/0061; C07C 43/205; C07C 69/76

(57) ABSTRACT

The present application relates to 4-sec-butoxy-1-benzaldehyde and related compounds, methods of making them, and methods of using them as flavor and fragrance ingredients in food, cosmetic, pharmaceutical, consumer, and other compositions and products.

### CROSS-REFERENCE TO RELATED APPLICATIONS

This application is an application filed under 35 U.S.C. § 111(a) which claims priority to, and the benefit of, U.S. Provisional Applic. Ser. No. 63/624,743, filed on Jan. 24, 2024, the contents of which are hereby incorporated by reference in its entirety.

### FIELD OF THE APPLICATION

The present application relates to 4-sec-butoxy-1-benzaldehyde and related compounds, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Toasted coconut and other nutty smells may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

Using a proprietary predictive method, the inventors have identified alkoxy-substituted benzene derivatives, and related compounds, as new ingredients useful in flavors and fragrances. Such compounds have not been specifically identified as a flavor or fragrance ingredient, nor have their smells or odors been previously described.

This application describes the surprising and unexpected olfactive qualities of alkoxy-substituted benzene derivatives, such as 4-sec-butoxy-1-benzaldehyde, and analogs and derivatives thereof, and their use as fragrance and flavor ingredients, and potential applications thereof.

### BRIEF SUMMARY

In a first aspect, the present disclosure provides a compound of Formula A:

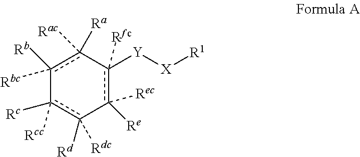

wherein:
Y is C₁₋₆alkylene or C₂₋₆alkenylene, or Y is absent;
X is —O—, —C(O)—, —OC(O)—, or —C(O))—, or X is absent;
one of R^{a}, R^{b}, R^{c}, R^{d}, and R^{e}, is OR², and the remaining four of R^{a}, R^{b}, R^{c}, R^{d}, and R^{e}, is independently a group R;
each of R^{ac}, R^{bc}, R^{cc}, R^{dc}, R^{ec}, and R^{fc}, if present, is independently selected from H, C₁₋₃alkyl (e.g., methyl), and OH;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is each independently selected from H, C₁₋₃alkyl (e.g., methyl), OH, and NH₂;
optionally provided that the compound is not 4-sec-butoxy-1-benzaldehyde.

In some embodiments the compound of Formula A is a compound of Formula I, Formula II, or Formula III.

In one embodiment of the first aspect, the application relates to compounds of Formula I:

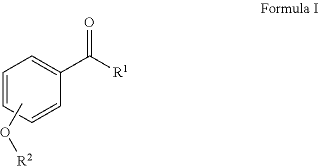

wherein:
R¹ is H, OH, optionally substituted C₁₋₆alkyl, optionally substituted C₃₋₆cycloalkyl, or OR³;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
optionally provided that the compound is not 4-sec-butoxy-1-benzaldehyde.

In some embodiments, the compound of Formula I is 4-sec-butoxy-1-benzaldehyde.

In another embodiment of the first aspect, the application relates to compounds of Formula II:

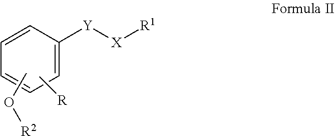

wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂;
optionally provided that the compound is not 4-sec-butoxy-1-benzaldehyde.

In another embodiment of the first aspect, the application relates to compounds of Formula III:

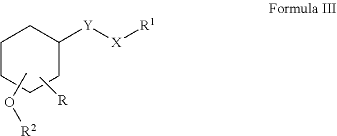

wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂.

In another aspect, the application relates to fragrance and flavor compositions comprising the compound of Formula I, Formula II, or Formula III, optionally comprising one or more additives, additional fragrance or flavor ingredients, or a combination of additives and fragrance or flavor ingredients. In some embodiments, the application relates to fragrance and flavor compositions comprising 4-sec-butoxy-1-benzaldehyde.

In another aspect, the application relates to products, such as consumer products, comprising such fragrance and flavor compositions comprising the compound of Formula I, Formula II, or Formula III, as herein provided, e.g., products comprising 4-sec-butoxy-1-benzaldehyde.

In another aspect, the present disclosure provides a method of making compounds of Formula I, Formula II, or Formula III.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that the compound 4-sec-butoxy-1-benzaldehyde has a unique and favorable aroma. It has thus been determined to be useful in imparting and providing desirable aromas and/or flavors to the products to which it is added. Other Compounds of Formula I, Formula II, and Formula III, are expected to likewise have pleasant or desirable flavors and/or aromas. Such Compounds are therefore potentially useful for products where the inclusion of a pleasing fragrance or flavor is desired, including, but not limited to, perfumes, household products, laundry products, personal care products, cosmetics, dental hygiene products, orally administered medications, and food products. The Compounds of Formula I, Formula II, and Formula III, may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula A:

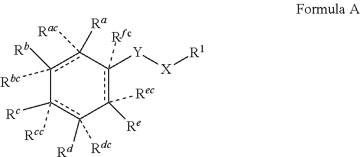

wherein:
Y is C₁₋₆alkylene or C₂₋₆alkenylene, or Y is absent;
X is —O—, —C(O)—, —OC(O)—, or —C(O))—, or X is absent; one of R^{a}, R^{b}, R^{c}, R^{d}, and R^{e}, is OR², and the remaining four of R^{a}, R^{b}, R^{c}, R^{d}, and R^{e}, is independently a group R;
each of R^{ac}, R^{bc}, R^{cc}, R^{dc}, R^{ec}, and R^{fc}, if present, is independently selected from H, C₁₋₃alkyl (e.g., methyl), and OH;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is each independently selected from H, C₁₋₃alkyl (e.g., methyl), OH, and NH₂;
optionally provided that the compound is not 4-sec-butoxy-1-benzaldehyde.

In some embodiments the compound of Formula A is a compound of Formula I, Formula II, or Formula III.

In the compound of Formula A, the six-membered ring

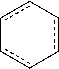

is either a phenyl ring or a cyclohexane ring, and that the dashed bonds are either absent or present. When the six-membered ring is a phenyl ring, it is substituted by the groups R^{a}, R^{b}, R^{c}, R^{d}, and R^{e}, as shown below. When the six-membered ring is a cyclohexane ring, it is substituted by the groups R^{a}, R^{b}, R^{c}, R^{d}, R^{e}, R^{ac}, R^{bc}, R^{cc}, R^{dc}, R^{ec}, and R^{fc}, as shown below:

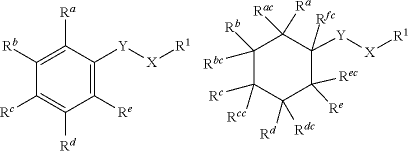

In a particular embodiment of the first aspect, the present disclosure provides a compound of Formula I:

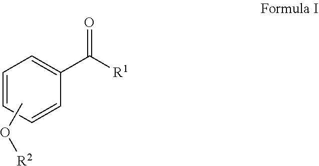

wherein:
R¹ is H, OH, optionally substituted C₁₋₆alkyl, optionally substituted C₃₋₆cycloalkyl, or OR³;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
optionally provided that the compound is not 4-sec-butoxy-1-benzaldehyde.

In further embodiments, of the first aspect, the present disclosure provides:
1.1 Compound of Formula I, wherein R¹ is H, OH, optionally substituted C₁₋₆alkyl, optionally substituted C₃₋₆cycloalkyl, or OR³;
1.2 Compound of Formula I, wherein R¹ is H;
1.3 Compound of Formula I, wherein R¹ is optionally substituted C₁₋₆alkyl;
1.4 Compound of Formula I, wherein R¹ is optionally substituted C₃₋₆cycloalkyl;
1.5 Compound of Formula I, wherein R¹ is OR³;
1.6 Compound of Formula I, or any of 1.1-1.5, wherein R² is optionally substituted C₁₋₃alkyl (e.g., C₁₋₅alkyl, C₁₋₇alkyl, C₁₋₉alkyl);
1.7 Compound of Formula I, or any of 1.1-1.5, wherein R² is linear C₁₋₃alkyl (e.g., C₁₋₅alkyl, C₁₋₇alkyl, C₁₋₉alkyl);
1.8 Compound of Formula I, or any of 1.1-1.5, wherein R² is selected from methyl, ethyl, propyl, n-butyl, n-pentyl, n-hexyl, n-heptyl, n-octyl, n-nonyl, and n-decyl;
1.9 Compound of Formula I, or any of 1.1-1.5, wherein R² is branched C₃₋₁₃alkyl (e.g., C₃₋₉alkyl, C₃₋₇alkyl, or C₃₋₅alkyl);
1.10 Compound of Formula I, or any of 1.1-1.5, wherein R² is selected from isopropyl, sec-butyl, isobutyl, tert-butyl, isopentyl, tert-pentyl, and tert-hexyl;
1.11 Compound of Formula I, or any of 1.1-1.5, wherein R² is unsubstituted;
1.12 Compound of Formula I, or any of 1.1-1.10, wherein R² is substituted by one or more groups selected from halo, hydroxy, and C₁₋₃alkoxy;
1.13 Compound of Formula I, or any of 1.1-1.5, wherein R² is substituted by one or more groups selected from C₁₋₃alkoxy (e.g., methoxy or ethoxy);
1.14 Compound of Formula I, or any of 1.1-1.13, wherein the —OR² group is positioned para to the —C(═O)R¹ group of the compound of Formula I;
1.15 Compound of Formula I, or any of 1.1-1.13, wherein the —OR² group is positioned meta to the —C(═O)R¹ group of the compound of Formula I
1.16 Compound of Formula I, or any of 1.1-1.13, wherein the —OR² group is positioned ortho to the —C(═O)R¹ group of the compound of Formula I;
1.17 Compound of Formula I, or any of 1.1-1.16, wherein R³ is C₁₋₆alkyl or C₃₋₆cycloalkyl;
1.18 Compound of Formula I, or any of 1.1-1.16, wherein R³ is C₁₋₆alkyl (e.g., methyl, ethyl, propyl, isopropyl, n-butyl, sec-butyl, isobutyl, tert-butyl, n-pentyl, or n-hexyl);
1.19 Compound of Formula I, or any of 1.1-1.16, wherein R³ is C₃₋₆cycloalkyl (e.g., cyclopropyl, cyclobutyl, cyclopentyl, or cyclohexyl);
1.20 Compound of Formula I, or any of 1.1-1.19 wherein the compound of Formula I is:

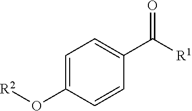

wherein R¹ and R² are as defined in any of the preceding formulae;
1.21 Compound of Formula I, or any of 1.1-1.19 wherein R¹ is H and R² is branched C₁₋₁₃alkyl;
1.22 Compound of Formula I, or any of 1.1-1.19 wherein the compound of Formula I is:

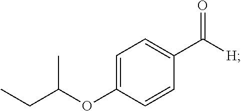

1.23 Compound of Formula I or any of 1.1-1.21, wherein the compound is not 4-sec-butoxy-1-benzaldehyde;
1.24 Any preceding compound, wherein the compound is the compound of Example 1, 2, 4, 58 or 59;
1.25 Any preceding compound, wherein the compound is the compound of Example 1, 2, or 4;
1.26 Any preceding compound, wherein the compound has a molecular weight selected from the range of to 130 to 600, or 130 to 500, or 130 to 450, or 130 to 400, or 130 to 350, or 130 to 300, or 130 to 250, or 130 to 200;
1.27 Any preceding compound wherein if the compound has an acidic or basic atom or functional group, then the compound is in the form of a salt, e.g., a base addition salt or an acid addition salt;
1.28 Any preceding compound, wherein the compound has a pleasing taste and/or aroma, e.g., as judged by a trained flavor or fragrance chemist or master perfumer (e.g., toasted coconut or other nutty aromas and/or tastes).

In another particular embodiment of the first aspect, the present disclosure provides a compound of Formula II:

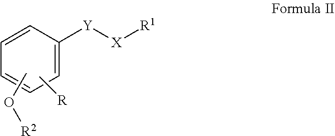

wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂;
optionally provided that the compound is not 4-sec-butoxy-1-benzaldehyde.

In further embodiments, of this embodiment, the present disclosure provides:
2.1. Compound of Formula II, wherein R¹ is H, OH, optionally substituted C₁₋₆alkyl, optionally substituted C₃₋₆cycloalkyl, or OR³;
2.2. Compound of Formula II, wherein R¹ is H;
2.3. Compound of Formula II, wherein R¹ is optionally substituted C₁₋₆alkyl (e.g., methyl, ethyl, propyl, isopropyl, n-butyl, sec-butyl, isobutyl, or tert-butyl);
2.4. Compound of Formula II, wherein R¹ is optionally substituted C₃₋₆cycloalkyl (e.g., cyclopropyl, 1-methylcyclopropyl, cyclobutyl, or 1-methylcyclobutyl);
2.5. Compound of Formula II, wherein R¹ is methyl or ethyl;
2.6. Compound of Formula II, wherein R¹ is OR³;
2.7. Compound of Formula II, or any of 2.1-1.5, wherein X— is —O;
2.8. Compound of Formula II, or any of 2.1-1.5, wherein X is —C(O)—;
2.9. Compound of Formula II, or any of 2.1-1.5, wherein X is —OC(O)—;
2.10. Compound of Formula II, or any of 2.1-1.5, wherein X is —C(O))—;
2.11. Compound of Formula II, or any of 2.1-1.5, wherein X is absent;
2.12. Compound of Formula II, or any of 2.1-2.11, wherein X—R¹ is —C(O)OH;
2.13. Compound of Formula II, or any of 2.1-2.11, wherein X—R¹ is —C(O)H;
2.14. Compound of Formula II, or any of 2.1-2.11, wherein X—R¹ is —C(O)R¹ and R¹ is optionally substituted C₁₋₆alkyl or optionally substituted C₃₋₆cycloalkyl;
2.15. Compound of Formula II, or any of 2.1-2.11, wherein X—R¹ is —C(O)OR¹ and R¹ is optionally substituted C₁₋₆alkyl or optionally substituted C₃₋₆cycloalkyl;
2.16. Compound of Formula II, or any of 2.1-2.11, wherein X—R¹ is —OC(O)R¹ and R¹ is optionally substituted C₁₋₆alkyl or optionally substituted C₃₋₆cycloalkyl;
2.17. Compound 2.14, 2.15, or 2.16, wherein R¹ is methyl, ethyl, propyl, isopropyl, n-butyl, sec-butyl, isobutyl, or tert-butyl;
2.18. Compound 2.14, 2.15, or 2.16, wherein R¹ is methyl or ethyl;
2.19. Compound of Formula II, or any of 2.1-2.11, wherein X—R¹ is —C(O)OR³;
2.20. Compound of Formula II, or any of 2.1-2.11, wherein X—R¹ is —OC(O)R³;
2.21. Compound of Formula II, or any of 2.1-2.11, wherein X—R¹ is —OR³;
2.22. Compound of Formula II, or any of 2.1-2.11, wherein X—R¹ is —OH;
2.23. Compound of Formula II, or any of 2.1-2.22, wherein Y is C₁₋₆alkylene, e.g., C₁₋₅alkylene, C₁₋₄alkylene, C₁₋₃alkylene, —CH₂—, —C₂-alkylene, C₃-alkylene, C₄-alkylene, C₅-alkylene, or C₆-alkylene;
2.24. Compound of Formula II, or any of 2.1-2.22, wherein Y is —CH₂—;
2.25. Compound of Formula II, or any of 2.1-2.22, wherein Y is —CH₂CH₂—, or —CH(CH₃);
2.26. Compound of Formula II, or any of 2.1-2.22, wherein Y is —CH₂CH₂CH₂—, CH(CH₃)CH₂—, —CH₂CH(CH₃)—, or —C(CH₃)₂—;
2.27. Compound of Formula II, or any of 2.1-2.22, wherein Y is —CH₂CH₂CH₂CH₂—, —CH(CH₃)CH₂CH₂—, —CH₂CH(CH₃)CH₂—, —CH₂CH₂CH(CH₃)—, —CH(CH₃)CH(CH₃)—, —CH₂C(CH₃)₂—, or —C(CH₃)₂CH₂—;
2.28. Compound of Formula II, or any of 2.1-2.22, wherein Y is —CH₂CH₂CH₂CH₂CH₂—, —CH₂CH(CH₃)CH₂CH₂—, —CH₂CH₂CH(CH₃)CH₂—, —CH₂CH₂CH₂CH(CH₃)—, —CH₂CH(CH₃)CH(CH₃)—, —CH(CH₃)CH₂CH₂CH₂—, —CH₂CH(CH₃)CH(CH₃)—, —CH(CH₃)CH(CH₃)CH₂—, —CH₂C(CH₃)₂CH₂—;
2.29. Compound of Formula II, or any of 2.1-2.22, wherein Y is C₂₋₆alkenylene, C₂₋₅alkenylene, C₂₋₄alkenylene, or C₂₋₃alkenylene;
2.30. Compound of Formula II, or any of 2.1-2.22, wherein Y is C₂alkenylene (i.e., —CH═CH—, or —C(═CH₂)—;
2.31. Compound of Formula II, or any of 2.1-2.22, wherein Y is C₃alkenylene (e.g., —CH₂CH═CH—, —CH═CHCH₂—, C(CH₃)═CH—, —CH═C(CH₃)—, —CH₂C(═CH₂)—, or —C(═CH₂)CH₂—;
2.32. Compound of Formula II, or any of 2.1-2.22, wherein Y is C₄alkenylene (e.g., —CH₂CH═CHCH₂—, —CH═CHCH₂CH₂—, —CH₂CH₂CH═CH—, CH₂C(CH₃)=CH—, —CH═C(CH₃)CH₂—, —C(CH₃)═C(CH₃)—, —CH₂CH₂C(═CH₂)—, or —C(═CH₂)CH₂CH₂—;
2.33. Compound of Formula II, or any of 2.1-2.22, wherein Y is absent;
2.34. Compound 2.33, wherein X is absent;
2.35. Compound 2.34, wherein R¹ is optionally substituted C₁₋₆alkyl (e.g., isopropyl, sec-butyl, isobutyl, or tert-butyl);
2.36. Compound of Formula II, or any of 2.1-1.5, wherein R² is optionally substituted C₁₋₁₃alkyl (e.g., C₁₋₅alkyl, C₁₋₇alkyl, C₁₋₉alkyl);
2.37. Compound of Formula II, or any of 2.1-1.5, wherein R² is linear C₁₋₁₃alkyl (e.g., C₁₋₅alkyl, C₁₋₇alkyl, C₁₋₉alkyl);
2.38. Compound of Formula II, or any of 2.1-1.5, wherein R² is selected from methyl, ethyl, propyl, n-butyl, n-pentyl, n-hexyl, n-heptyl, n-octyl, n-nonyl, and n-decyl;
2.39. Compound of Formula II, or any of 2.1-1.5, wherein R² is branched C₃₋₁₃alkyl (e.g., C₃₋₉alkyl, C₃₋₇alkyl, or C₃₋₅alkyl);
2.40. Compound of Formula II, or any of 2.1-1.5, wherein R² is selected from isopropyl, sec-butyl, isobutyl, tert-butyl, isopentyl, tert-pentyl, and tert-hexyl;
2.41. Compound of Formula II, or any of 2.1-1.5, wherein R² is selected from isopropyl, sec-butyl, isobutyl, and tert-butyl;
2.42. Compound of Formula II, or any of 2.1-1.5, wherein R² is selected from isopropyl, sec-butyl, and isobutyl;
2.43. Compound of Formula II, or any of 2.1-1.5, wherein R² is selected from sec-butyl and isobutyl;
2.44. Compound of Formula II, or any of 2.1-1.5, wherein R² is sec-butyl;
2.45. Compound of Formula II, or any of 2.1-2.44, wherein R² is unsubstituted;
2.46. Compound of Formula II, or any of 2.1-2.44, wherein R² is substituted by one or more groups selected from halo, hydroxy, and C₁₋₃alkoxy;
2.47. Compound of Formula II, or any of 2.1-2.44, wherein R² is substituted by one or more groups selected from C₁₋₃alkoxy (e.g., methoxy or ethoxy);
2.48. Compound of Formula II, or any of 2.1-2.47, wherein the —OR² group is positioned para to the —Y—X—R¹ group of the compound of Formula II;
2.49. Compound of Formula II, or any of 2.1-2.47, wherein the —OR² group is positioned meta to the —Y—X—R¹ group of the compound of Formula II;
2.50. Compound of Formula II, or any of 2.1-2.47, wherein the —OR² group is positioned ortho to the —Y—X—R¹ group of the compound of Formula II;
2.51. Compound of Formula II, or any of 2.1-2.50, wherein R is C₁₋₃alkyl (e.g., methyl);
2.52. Compound of Formula II, or any of 2.1-2.50, wherein R is OH;
2.53. Compound of Formula II, or any of 2.1-2.50, wherein R is NH₂;
2.54. Compound of Formula II, or any of 2.46-2.53, wherein R is positioned ortho to the —Y—X—R¹ group of the compound of Formula II;
2.55. Compound of Formula II, or any of 2.51-2.53, wherein R is positioned meta to the —Y—X—R¹ group of the compound of Formula II;
2.56. Compound of Formula II, or any of 2.51-2.53, wherein R is positioned para to the —Y—X—R¹ group of the compound of Formula II;
2.57. Compound of Formula II, or any of 2.1-2.56, wherein R³ is C₁₋₆alkyl (e.g., methyl, ethyl, propyl, isopropyl, n-butyl, sec-butyl, isobutyl, tert-butyl, n-pentyl, or n-hexyl);
2.58. Compound of Formula II, or any of 2.1-2.56, wherein R³ is C₃₋₆cycloalkyl (e.g., cyclopropyl, cyclobutyl, cyclopentyl, or cyclohexyl);
2.59. Compound of Formula II, or any of 2.1-2.58, wherein the compound of Formula II is:

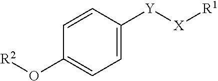

wherein Y, X, R¹ and R² are as defined in any of the preceding formulae;
2.60. Compound of Formula II, or any of 2.1-2.58, wherein the compound of Formula II is:

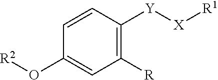

wherein Y, X, R, R¹ and R² are as defined in any of the preceding formulae;
2.61. Compound of Formula II, or any of 2.1-2.58, wherein the compound of Formula II is:

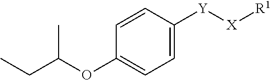

wherein Y, X, and R¹ are as defined in any of the preceding formulae;
2.62. Compound of Formula II, or any of 2.1-2.58, wherein the compound of Formula II is:

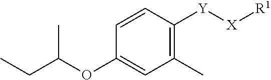

wherein Y, X, and R¹ are as defined in any of the preceding formulae;
2.63. Compound of Formula 2.61, 2.62, 2.63, or 2.64, wherein Y—X—R is —C(O)H, —C₁₋₅alkylene-C(O)H, or C₂₋₅alkenylene-C(O)H;
2.64. Compound of Formula 2.61, 2.62, 2.63, or 2.64, wherein Y—X—R is —C(O)R¹, —C₁₋₅alkylene-C(O)R¹, or C₂₋₅alkenylene-C(O)R¹; and R¹ is optionally substituted C₁₋₃alkyl (e.g., methyl or ethyl);
2.65. Compound of Formula 2.61, 2.62, 2.63, or 2.64, wherein Y—X—R is —C(O)OR¹, —C₁₋₅alkylene-C(O)OR¹, or C₂₋₅alkenylene-C(O)OR¹; and R¹ is optionally substituted C₁₋₃alkyl (e.g., methyl or ethyl);
2.66. Compound of Formula 2.61, 2.62, 2.63, or 2.64, wherein Y—X—R is —OC(O)R¹, —C₁₋₅alkylene-OC(O)R¹, or C₂₋₅alkenylene-OC(O)R¹; and R¹ is optionally substituted C₁₋₃alkyl (e.g., methyl or ethyl);
2.67. Compound of Formula 2.61, 2.62, 2.63, or 2.64, wherein Y—X—R is —C₁₋₅alkylene-OH, or C₂₋₅alkenylene-OH;
2.68. Compound of Formula 2.61, 2.62, 2.63, or 2.64, wherein Y—X—R is —C₁₋₅alkylene-OR³, or C₂₋₅alkenylene-OR³; and R³ C₁₋₃alkyl (e.g., methyl or ethyl);
2.69. Compound of Formula II or any of 2.1-2.68, wherein the compound is not 4-sec-butoxy-1-benzaldehyde;
2.70. Any preceding compound, wherein the compound has a molecular weight selected from the range of to 130 to 600, or 130 to 500, or 130 to 450, or 130 to 400, or 130 to 350, or 130 to 300, or 130 to 250, or 130 to 200;
2.71. Any preceding compound, wherein the double bond of the alkylene group is oriented with (E) stereochemistry;
2.72. Any preceding compound, wherein the double bond of the alkylene group is oriented with (Z) stereochemistry;
2.73. Any preceding compound, wherein the compound is selected from any one or more of the compounds of Examples 1-4, 6-10, 12-23, 25-29, 31, 33, 36-38, 40, 42, 43, 47-51, 54, 55, and 57-59, as defined herein;
2.74. Any preceding compound, wherein the compound is selected from any one or more of the compounds of Examples 2, 6, 17, 49, 50, 51, and 55, as defined herein
2.75. Any preceding compound wherein if the compound has an acidic or basic atom or functional group, then the compound is in the form of a salt, e.g., a base addition salt or an acid addition salt;
2.76. Any preceding compound, wherein the compound has a pleasing taste and/or aroma, e.g., as judged by a trained flavor or fragrance chemist or master perfumer (e.g., toasted coconut or other nutty aromas and/or tastes).

In another particular embodiment of the first aspect, the present disclosure provides a compound of Formula III:

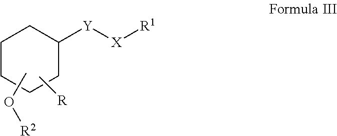

wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³; provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂.

In further embodiments, of this embodiment, the present disclosure provides:
3.1. Compound of Formula III, wherein R¹ is H, OH, optionally substituted C₁₋₆alkyl, optionally substituted C₃₋₆cycloalkyl, or OR³;
3.2. Compound of Formula III, wherein R¹ is H;
3.3. Compound of Formula III, wherein R¹ is optionally substituted C₁₋₆alkyl (e.g., methyl, ethyl, propyl, isopropyl, n-butyl, sec-butyl, isobutyl, or tert-butyl);
3.4. Compound of Formula III, wherein R¹ is optionally substituted C₃₋₆cycloalkyl (e.g., cyclopropyl, 1-methylcyclopropyl, cyclobutyl, or 1-methylcyclobutyl);
3.5. Compound of Formula III, wherein R¹ is methyl or ethyl;
3.6. Compound of Formula III, wherein R¹ is OR³;
3.7. Compound of Formula III, or any of 3.1-1.5, wherein X— is —O;
3.8. Compound of Formula III, or any of 3.1-1.5, wherein X is —C(O)—;
3.9. Compound of Formula III, or any of 3.1-1.5, wherein X is —OC(O)—;
3.10. Compound of Formula III, or any of 3.1-1.5, wherein X is —C(O))—;
3.11. Compound of Formula III, or any of 3.1-1.5, wherein X is absent;
3.12. Compound of Formula III, or any of 3.1-3.11, wherein X—R¹ is —C(O)OH;
3.13. Compound of Formula III, or any of 3.1-3.11, wherein X—R¹ is —C(O)H;
3.14. Compound of Formula III, or any of 3.1-3.11, wherein X—R¹ is —C(O)R¹ and R¹ is optionally substituted C₁₋₆alkyl or optionally substituted C₃₋₆cycloalkyl;
3.15. Compound of Formula III, or any of 3.1-3.11, wherein X—R¹ is —C(O)OR¹ and R¹ is optionally substituted C₁₋₆alkyl or optionally substituted C₃₋₆cycloalkyl;
3.16. Compound of Formula III, or any of 3.1-3.11, wherein X—R¹ is —OC(O)R¹ and R¹ is optionally substituted C₁₋₆alkyl or optionally substituted C₃₋₆cycloalkyl;
3.17. Compound 3.14, 3.15, or 3.16, wherein R¹ is methyl, ethyl, propyl, isopropyl, n-butyl, sec-butyl, isobutyl, or tert-butyl;
3.18. Compound 3.14, 3.15, or 3.16, wherein R¹ is methyl or ethyl;
3.19. Compound of Formula III, or any of 3.1-3.11, wherein X—R¹ is —C(O)OR³;
3.20. Compound of Formula III, or any of 3.1-3.11, wherein X—R¹ is —OC(O)R³;
3.21. Compound of Formula III, or any of 3.1-3.11, wherein X—R¹ is —OR³;
3.22. Compound of Formula III, or any of 3.1-3.11, wherein X—R¹ is —OH;
3.23. Compound of Formula III, or any of 3.1-3.22, wherein Y is C₁₋₆alkylene, e.g., C₁₋₅alkylene, C₁₋₄alkylene, C₁₋₃alkylene, —CH₂—, —C₂-alkylene, C₃-alkylene, C₄-alkylene, Cs-alkylene, or C₆-alkylene;
3.24. Compound of Formula III, or any of 3.1-3.22, wherein Y is —CH₂—;
3.25. Compound of Formula III, or any of 3.1-3.22, wherein Y is —CH₂CH₂—, or —CH(CH₃);
3.26. Compound of Formula III, or any of 3.1-3.22, wherein Y is —CH₂CH₂CH₂—, CH(CH₃)CH₂—, —CH₂CH(CH₃)—, or —C(CH₃)₂—;
3.27. Compound of Formula III, or any of 3.1-3.22, wherein Y is —CH₂CH₂CH₂CH₂—, —CH(CH₃)CH₂CH₂—, —CH₂CH(CH₃)CH₂—, —CH₂CH₂CH(CH₃)—, —CH(CH₃)CH(CH₃)—, —CH₂C(CH₃)₂—, or —C(CH₃)₂CH₂—;
3.28. Compound of Formula III, or any of 3.1-3.22, wherein Y is —CH₂CH₂CH₂CH₂CH₂—, —CH₂CH(CH₃)CH₂CH₂—, —CH₂CH₂CH(CH₃)CH₂—, —CH₂CH₂CH₂CH(CH₃)—, —CH₂CH(CH₃)CH(CH₃)—, —CH(CH₃)CH₂CH₂CH₂—, —CH₂CH(CH₃)CH(CH₃)—, —CH(CH₃)CH(CH₃)CH₂—, —CH₂C(CH₃)₂CH₂—;
3.29. Compound of Formula III, or any of 3.1-3.22, wherein Y is C₂₋₆alkenylene, C₂₋₅alkenylene, C₂₋₄alkenylene, or C₂₋₃alkenylene;
3.30. Compound of Formula III, or any of 3.1-3.22, wherein Y is C₂alkenylene (i.e., —CH═CH—, or —C(═CH₂)—;
3.31. Compound of Formula III, or any of 3.1-3.22, wherein Y is C₃alkenylene (e.g., —CH₂CH═CH—, —CH═CHCH₂—, C(CH₃)═CH—, —CH═C(CH₃)—, —CH₂C(═CH₂)—, or —C(═CH₂)CH₂—;
3.32. Compound of Formula III, or any of 3.1-3.22, wherein Y is C₄alkenylene (e.g., —CH₂CH═CHCH₂—, —CH═CHCH₂CH₂—, —CH₂CH₂CH═CH—, CH₂C(CH₃)═CH—, —CH═C(CH₃)CH₂—, —C(CH₃)═C(CH₃)—, —CH₂CH₂C(═CH₂)—, or —C(═CH₂)CH₂CH₂—;
3.33. Compound of Formula III, or any of 3.1-3.22, wherein Y is absent;
3.34. Compound 3.33, wherein X is absent;
3.35. Compound 3.34, wherein R¹ is optionally substituted C₁₋₆alkyl (e.g., isopropyl, sec-butyl, isobutyl, or tert-butyl);
3.36. Compound of Formula III, or any of 3.1-1.5, wherein R² is optionally substituted C₁₋₁₃alkyl (e.g., C₁₋₅alkyl, C₁₋₇alkyl, C₁₋₉alkyl);
3.37. Compound of Formula III, or any of 3.1-1.5, wherein R² is linear C₁₋₃alkyl (e.g., C₁₋₅alkyl, C₁₋₇alkyl, C₁₋₉alkyl);
3.38. Compound of Formula III, or any of 3.1-1.5, wherein R² is selected from methyl, ethyl, propyl, n-butyl, n-pentyl, n-hexyl, n-heptyl, n-octyl, n-nonyl, and n-decyl;
3.39. Compound of Formula III, or any of 3.1-1.5, wherein R² is branched C₃₋₁₃alkyl (e.g., C₃₋₉alkyl, C₃₋₇alkyl, or C₃₋₅alkyl);
3.40. Compound of Formula III, or any of 3.1-1.5, wherein R² is selected from isopropyl, sec-butyl, isobutyl, tert-butyl, isopentyl, tert-pentyl, and tert-hexyl;
3.41. Compound of Formula III, or any of 3.1-1.5, wherein R² is selected from isopropyl, sec-butyl, isobutyl, and tert-butyl;
3.42. Compound of Formula III, or any of 3.1-1.5, wherein R² is selected from isopropyl, sec-butyl, and isobutyl;
3.43. Compound of Formula III, or any of 3.1-1.5, wherein R² is selected from sec-butyl and isobutyl;
3.44. Compound of Formula III, or any of 3.1-1.5, wherein R² is sec-butyl;
3.45. Compound of Formula III, or any of 3.1-3.44, wherein R² is unsubstituted;
3.46. Compound of Formula III, or any of 3.1-3.44, wherein R² is substituted by one or more groups selected from halo, hydroxy, and C₁₋₃alkoxy;
3.47. Compound of Formula III, or any of 3.1-3.44, wherein R² is substituted by one or more groups selected from C₁₋₃alkoxy (e.g., methoxy or ethoxy);
3.48. Compound of Formula III, or any of 3.1-3.47, wherein the —OR² group is positioned in a 1,4-position to the —Y—X—R¹ group of the compound of Formula III;
3.49. Compound of Formula III, or any of 3.1-3.47, wherein the —OR² group is positioned in a 1,3-position to the —Y—X—R¹ group of the compound of Formula III;
3.50. Compound of Formula III, or any of 3.1-3.47, wherein the —OR² group is positioned in a 1,2-position to the —Y—X—R¹ group of the compound of Formula III;
3.51. Compound of Formula III, or any of 3.1-3.50, wherein R is C₁₋₃alkyl (e.g., methyl);
3.52. Compound of Formula III, or any of 3.1-3.50, wherein R is OH;
3.53. Compound of Formula III, or any of 3.1-3.50, wherein R is NH₂;
3.54. Compound of Formula III, or any of 3.46-3.53, wherein R is positioned in a 1,2-position to the —Y—X—R¹ group of the compound of Formula III;
3.55. Compound of Formula III, or any of 3.51-3.53, wherein R is positioned in a 1,3-position to the —Y—X—R¹ group of the compound of Formula III;
3.56. Compound of Formula III, or any of 3.51-3.53, wherein R is positioned in a 1,3-position to the —Y—X—R¹ group of the compound of Formula III;
3.57. Compound of Formula III, or any of 3.1-3.56, wherein R³ is C₁₋₆alkyl (e.g., methyl, ethyl, propyl, isopropyl, n-butyl, sec-butyl, isobutyl, tert-butyl, n-pentyl, or n-hexyl);
3.58. Compound of Formula III, or any of 3.1-3.56, wherein R³ is C₃₋₆cycloalkyl (e.g., cyclopropyl, cyclobutyl, cyclopentyl, or cyclohexyl);
3.59. Compound of Formula III, or any of 3.1-3.58, wherein the compound of Formula III is:

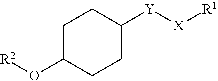

wherein Y, X, R¹ and R² are as defined in any of the preceding formulae;
3.60. Compound of Formula III, or any of 3.1-3.58, wherein the compound of Formula III is:

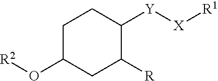

wherein Y, X, R, R¹ and R² are as defined in any of the preceding formulae;
3.61. Compound of Formula III, or any of 3.1-3.58, wherein the compound of Formula III is:

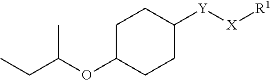

wherein Y, X, and R¹ are as defined in any of the preceding formulae;
3.62. Compound of Formula III, or any of 3.1-3.58, wherein the compound of Formula III is:

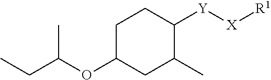

wherein Y, X, and R¹ are as defined in any of the preceding formulae;
3.63. Compound of Formula 3.61, 3.62, 3.63, or 3.64, wherein Y—X—R is —C(O)H, —C₁₋₅alkylene-C(O)H, or C₂₋₅alkenylene-C(O)H;
3.64. Compound of Formula 3.61, 3.62, 3.63, or 3.64, wherein Y—X—R is —C(O)R¹, —C₁₋₅alkylene-C(O)R¹, or C₂₋₅alkenylene-C(O)R¹; and R¹ is optionally substituted C₁₋₃alkyl (e.g., methyl or ethyl);
3.65. Compound of Formula 3.61, 3.62, 3.63, or 3.64, wherein Y—X—R is —C(O)OR¹, —C₁₋₅alkylene-C(O)OR¹, or C₂₋₅alkenylene-C(O)OR¹; and R¹ is optionally substituted C₁₋₃alkyl (e.g., methyl or ethyl);
3.66. Compound of Formula 3.61, 3.62, 3.63, or 3.64, wherein Y—X—R is —OC(O)R¹, —C₁₋₅alkylene-OC(O)R¹, or C₂₋₅alkenylene-OC(O)R¹; and R¹ is optionally substituted C₁₋₃alkyl (e.g., methyl or ethyl);
3.67. Compound of Formula 3.61, 3.62, 3.63, or 3.64, wherein Y—X—R is —C₁₋₅alkylene-OH, or C₂₋₅alkenylene-OH;
3.68. Compound of Formula 3.61, 3.62, 3.63, or 3.64, wherein Y—X—R is —C₁₋₅alkylene-OR³, or C₂₋₅alkenylene-OR³; and R³ C₁₋₃alkyl (e.g., methyl or ethyl);
3.69. Compound of Formula III or any of 3.1-3.68, wherein the —OR² group is positioned cis to the —Y—X—R¹ group of the compound of Formula III, with reference to the plane of the cyclohexane ring;
3.70. Compound of Formula III or any of 3.1-3.68, wherein the —OR² group is positioned trans to the —Y—X—R¹ group of the compound of Formula III, with reference to the plane of the cyclohexane ring;
3.71. Compound of Formula III or any of 3.1-3.68, wherein the —R group is positioned cis to the —Y—X—R¹ group of the compound of Formula III, with reference to the plane of the cyclohexane ring;
3.72. Compound of Formula III or any of 3.1-3.68, wherein the —R group is positioned trans to the —Y—X—R¹ group of the compound of Formula III, with reference to the plane of the cyclohexane ring;
3.73. Any preceding compound, wherein the double bond of the alkylene group is oriented with (E) stereochemistry;
3.74. Any preceding compound, wherein the double bond of the alkylene group is oriented with (Z) stereochemistry;
3.75. Any preceding compound, wherein the compound has a molecular weight selected from the range of to 130 to 600, or 130 to 500, or 130 to 450, or 130 to 400, or 130 to 350, or 130 to 300, or 130 to 250, or 130 to 200;
3.76. Any preceding compound, wherein the compound is selected from any one or more of the compounds of Examples 5, 11, 24, 30, 32, 34, 35, 39, 41, 44, 45, 46, 52, 53, 56, 60, or 61, as defined herein;
3.77. Any preceding compound wherein if the compound has an acidic or basic atom or functional group, then the compound is in the form of a salt, e.g., a base addition salt or an acid addition salt;
3.78. Any preceding compound, wherein the compound has a pleasing taste and/or aroma, e.g., as judged by a trained flavor or fragrance chemist or master perfumer (e.g., toasted coconut or other nutty aromas and/or tastes).

In a second aspect, the present disclosure provides a flavor composition and/or a fragrance composition (Composition 1) comprising a Compound of Formula A, a Compound of Formula I, or any of 1.1-1.28, a Compound of Formula II, or any of 2.1-2.76, or a Compound of Formula III, or any of 3.1-3.78, each in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound of Formula A, Compound of Formula I, or any of 1.1-1.28, Compound of Formula II, or any of 2.1-2.76, or Compound of Formula III, or any of 3.1-3.78, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula A, a Compound of Formula I, or any of 1.1-1.28, a Compound of Formula II, or any of 2.1-2.76, or a Compound of Formula III, or any of 3.1-3.78,) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredients in the composition and the concentration of the composition used in the product.

As used herein, the term “flavor composition” means a mixture of flavor ingredients (e.g., including a Compound of Formula A, or a Compound of Formula I, II, or III) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formulas A, I, II, or III which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, α-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dimethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang lignaloe, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)—N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea, EDTA, polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a fourth aspect, the present disclosure provides a product which comprises Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage ±5%, ±4%, ±3%, ±2.5%, ±2%, ±1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-penyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. Exemplary C₁₋₆-alkyl groups include methyl, ethyl, n-propyl, isopropyl, n-butyl, isobutyl, sec-butyl, tert-butyl. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl and allyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

As used herein, the term “heteroaryl” means an aromatic free radical having 5 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### EXAMPLES

Compounds of the present disclosure can be made according to known methods published in the art.

4-sec-butoxy-1-benzaldehyde is obtained and its olfactory qualities are studied. Both the neat liquid compound and a 10% dilution of the compound in ethanol are examined by an experienced fragrance chemist (a master perfumer). It is found that the compound exhibits a favorable odor profile.

### Synthetic Examples

The Compounds of Examples 1 to 56 and 60-61 may be prepared according to the procedures described hereinbelow and analogous procedures thereto. The Compounds of Examples 57-59 were purchased from a commercial supplier.

¹H-NMR spectra were recorded at 400 MHz on a Bruker Avance AV-I-400 instrument or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

### Purification Method Information:

Method I: Apparatus: Sepiatec Prep 250 SFC; Column: Phenomenex Lux i-Cellulose-5 (250×21.2 mm, 5 m); Column temp: 40° C.; Flow: 100 ml/min; ABPR: 120 bar; Eluent A: CO2, Eluent B: 20 mM Ammonia in Methanol; Isocratic method: 50% B for 8 min; Detection: UV 210; Collection: Timed.

### LCMS and GCMS Analytical Method Information:

LCMS Method 2: UPLC: Waters I-Class, Acq. Method: UPLC_AN_BASE, Column: XSelect CSH C18 XP (50×2.1 mm 2.5 μm), Flow: 0.6 ml/min; Column temp: 25° C., Eluent A: 10 mM ammonium bicarbonate in water (pH 9.5), Eluent B: acetonitrile, Gradient: t=0 min 5% B, t=2 min 98% B, t=2.7 min 98% B, Post-time: 0.3 min, Detection PDA: 210-320 nm.

LCMS Method 4: Acq. Method: U_AN_ACID, System: Agilent 1290 with SQ-MSD, Column: XSelect CSH XP C18 (50×2.1 mm, 2.5μ), Flow: 0.8 ml/min Column temp 40° C., Eluent A: 0.1% Formic acid in Water, Eluent B: 0.1% Formic acid in Acetonitrile, Lin. Gradient: t=0 min 5% B, t=0.5 min 5% B, t=4.5 min 98% B; t=5 min 98% B, Post-time: 0.5 min, Detection: DAD (210-320 nm, 215 nm), Detection: PDA (210-320 nm), Detection: MSD (ESI pos/neg) mass range 90-1500.

LCMS Method 5: Acq. Method: UPLC_AN_BASE, Column: XSelect CSH C18 XP (50×2.1 mm 2.5 μm), Flow: 0.6 ml/min; Column temp: 25° C., Eluent A: 10 mM ammonium bicarbonate in water (pH=9.5), Eluent B: 100% acetonitrile, Gradient: t=0 min 5% B, t=2 min 98% B, t=2.7 min 98% B, Post-time: 0.3 min, Detection PDA: 210-320 nm, Detection ELSD: gas pressure 40 psi, drift tube temp: 50° C.

GCMS Method 8: G40-300_M350, Instrument: Agilent 8890, G7081B 5977B MSD (ELI-positive, Det. temp.: 280° C.) Mass range 50-350, Detection FID/Det. temp: 325° C., Column: Agilent DB-5MS (20 m ID 180 μm, df 0.18 μm), Average velocity: 39 cm/s, Injection vol: 1 μl, Injector temp: 250° C., Split ratio: 100/1, Carrier gas: He; Initial temp: 40° C., Initial time: 0.5 min, Solvent delay: 1.1 min, Rate 120° C./min, 115° C.; 110° C./min, 175° C.; 80° C./min, Final temp 300° C., Hold time 2.0 min.

LCMS Method 9: Method: SC_ACID.M, Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gas flow: 1.3 ml/min), Column: XSelect CSH C18 (30×2.1 mm 3.5μ) Flow: 1 ml/min, Column temp.: 40° C., Eluent A: 0.1% Formic acid in Water, Eluent B: 0.1% Formic acid in Acetonitrile, Gradient: t=0 min 5% B, t=1.6 min 98% B, t=3 min 98% B, Post-run: 1.3 min. Source: API-ES, Capillary voltage: 3000 V, Drying gas flow: 13 L/min, Nebulizer Pressure 60 psig, Drying Gas Temp: 350° C., Fragmentor 70.

LCMS Method 10: Acq. Method: SC_BASE.M, Instrument: Agilent 1260 Infinity, 1260 G1312B Bin. Pump, 1260 G1367E WPS, 1260 TCC G1316A Column Comp. 1260 G1315C DAD (210-320 nm, 210 and 220 nm), PDA (210-320 nm), G6130B MSD ESI pos/neg (mass range 100-1000), Column: Waters XSelect CSH C18 (30×2.1 mm 3.5μ), Flow: 1 ml/min; Column Temp: 25° C., Eluent A: 10 mM ammonium bicarbonate in water (pH 9), Eluent B: Acetonitrile, Gradient: t=0 min 5% B, t=1.6 min 98% B, t=3 min 98% B, Post-run: 1.3 min. Source: API-ES, Capillary voltage: 3000 V, Drying gas flow: 13 L/min, Nebulizer Pressure 60 psig, Drying Gas Temp: 350° C., Fragmentor 70.

### Example 1: 1-(4-(sec-butoxy)phenyl)ethan-1-one

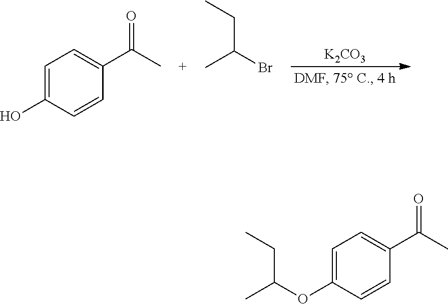

To 1-(4-hydroxyphenyl)ethan-1-one (5.60 g, 1 Eq, 41.1 mmol) in DMF (30 mL) were added 2-bromobutane (6.74 mL, 1.5 Eq, 61.7 mmol) and potassium carbonate (11.4 g, 2 Eq, 82.3 mmol) and the mixture was heated at 75° C. for 4 h. The mixture was allowed to cool to room temperature, diluted with water and DCM and layers separated. The aqueous was extracted twice with DCM and the combined organics were washed with brine, dried over Na₂SO₄, filtered and concentrated. Purification by flash column chromatography (SiO₂, 0-75% EtOAc in heptane) afforded 1-(4-(sec-butoxy)phenyl)ethan-1-one (6.26 g, 32.6 mmol, 79% yield). LCMS: Method 5, 1.63 min, M+H=193.1; calcd. 193.258, GCMS: Method 8, 2.80 min, 192.1; calcd. 192.1, ¹H NMR (400 MHz, CDCl₃) δ 7.95-7.87 (m, 2H), 6.94-6.85 (m, 2H), 4.40 (h, J=6.1 Hz, 1H), 2.55 (s, 3H), 1.84-1.60 (m, 2H), 1.32 (d, J=6.1 Hz, 3H), 0.98 (t, J=7.5 Hz, 3H).

### Example 2: 4-(sec-butoxy)benzaldehyde

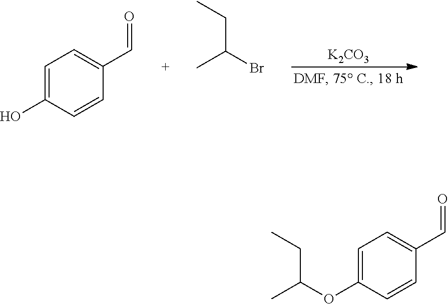

To a 4-hydroxybenzaldehyde (12.00 g, 1 Eq, 98.26 mmol) solution in DMF (49.13 mL) were added potassium carbonate (27.16 g, 2 Eq, 196.5 mmol) and 2-bromobutane (20.20 g, 16.09 mL, 1.5 Eq, 147.4 mmol) and the mixture was heated at 75° C. for 18 h. The reaction was stopped, and the mixture was diluted with water and DCM and layers were separated. The aqueous phase was extracted twice more with DCM and the combined organics were washed with brine, dried over Na₂SO₄, filtered and concentrated. Purification by flash column chromatography (SiO₂, 0-25% EtOAc in MBTE), affording 4-(sec-butoxy)benzaldehyde (15.37 g, 86.24 mmol, 88% yield) as yellow oil. LCMS: Method 9, 1.92 min, M+H=179.2; calcd. 178.099. ¹H NMR (400 MHz, CDCl₃) δ 9.87 (s, 1H), 7.84-7.79 (m, 2H), 7.00-6.95 (m, 2H), 4.43 (h, J=6.0 Hz, 1H), 1.84-1.72 (m, 1H), 1.72-1.61 (m, 1H), 1.34 (d, J=6.1 Hz, 3H), 0.99 (t, J=7.5 Hz, 3H).

### Example 3: 4-(sec-butoxy)-2-methylbenzaldehyde

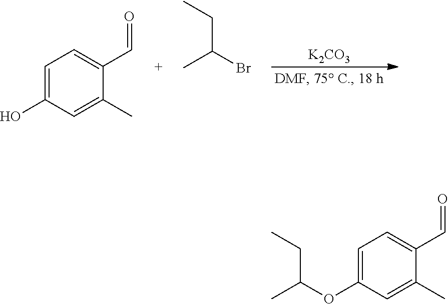

To 4-hydroxy-2-methylbenzaldehyde (15.0 g, 1 Eq, 110 mmol) in DMF (55 mL) were added 2-bromobutane (18.0 mL, 1.5 Eq, 165 mmol) and potassium carbonate (30.5 g, 2 Eq, 220 mmol) and the mixture was heated at 75° C. for 18 h. The mixture was allowed to cool to room temperature, diluted with water and DCM and layers separated. The aqueous was extracted twice with DCM and the combined organics were washed with brine, dried over Na₂SO₄, filtered and concentrated. Purification by flash column chromatography (SiO₂, 0-25% EtOAc in MTBE) afforded 4-(sec-butoxy)-2-methylbenzaldehyde (18.48 g, 96.12 mmol, 87% yield). LCMS: Method 4, 3.08 min, M+H=193; calcd. 193.258, GCMS: Method 8, 2.82 min, 192.1; calcd. 192.2, 1H NMR (400 MHz, CDCl³) δ 10.09 (s, 1H), 7.73 (d, J=8.6 Hz, 1H), 6.81 (dd, J=8.6, 2.6 Hz, 1H), 6.72 (d, J=2.5 Hz, 1H), 4.41 (h, J=6.0 Hz, 1H), 2.63 (s, 3H), 1.84-1.71 (m, 1H), 1.71-1.58 (m, 1H), 1.32 (d, J=6.1 Hz, 3H), 0.98 (t, J=7.5 Hz, 3H).

### Example 4: ethyl 4-(sec-butoxy)benzoate

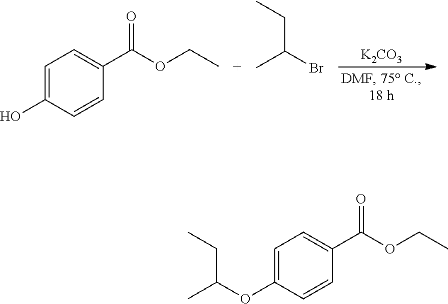

To ethyl paraben (2.0 g, 1 Eq, 12 mmol) in DMF (6.0 mL) were added 2-bromobutane (2.0 mL, 1.5 Eq, 18 mmol) and potassium carbonate (3.3 g, 2 Eq, 24 mmol) and the mixture was heated at 75° C. for 18 h. The mixture was allowed to cool to room temperature, diluted with water and DCM and layers separated. The aqueous was extracted twice with DCM and the combined organics were washed with brine, dried over Na₂SO₄, filtered and concentrated. Purification by flash column chromatography (SiO₂, 0-50% EtOAc in heptane) afforded ethyl 4-(sec-butoxy)benzoate (2.0 g, 9.0 mmol, 75% yield). LCMS: Method 4, 3.39 min, M+H=223.0; calcd. 223.284, GCMS: Method 8, 2.94 min, 222.1; calcd. 222.2, ¹H NMR (400 MHz, CDCl₃) δ 8.06-7.85 (m, 2H), 6.97-6.76 (m, 2H), 4.45-4.27 (m, 3H), 1.85-1.70 (m, 1H), 1.67-1.56 (m, 1H), 1.37 (t, J=7.1 Hz, 3H), 1.31 (d, J=6.1 Hz, 3H), 0.98 (t, J=7.4 Hz, 3H).

### Example 5: methyl 4-(sec-butoxy)cyclohexane-1-carboxylate

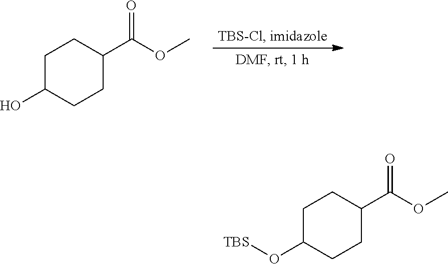

### Step 1: methyl 4-((tert-butyldimethylsilyl)oxy)cyclohexane-1-carboxylate

To a solution of methyl 4-hydroxycyclohexanecarboxylate (2.0 g, 1 Eq, 13 mmol) and imidazole (1.9 g, 1.8 mL, 2.2 Eq, 28 mmol) in DMF (8.4 mL) was added tert-butyldimethyl chlorosilane (2.1 mL, 1 Eq, 13 mmol) and the mixture was stirred at room temperature for 1 h. 10% citric acid (50.0 mL) was added and the mixture was extracted with diethyl ether. Combined organics were washed with water, brine, dried over Na₂SO₄, filtered and concentrated to give methyl 4-((tert-butyldimethylsilyl)oxy)cyclohexane-1-carboxylate (3.3 g, 12 mmol, 96% yield) (33:67 mixture of isomers). GCMS: Method 8, 2.82 and 2.85 min, 272.1; calcd. 272.4, ¹H NMR (400 MHz, CDCl₃) δ 3.61-3.60 (m, 3H), 3.54-3.48 (m, 1H), 2.28-2.16 (m, 1H), 1.92-1.81 (m, 2H), 1.65-1.56 (m, 1H), 1.48-1.37 (m, 2H), 1.30-1.20 (m, 1H), 0.86 (s, 2H), 0.83 (d, J=2.0 Hz, 9H), 0.08-0.12 (m, 6H).

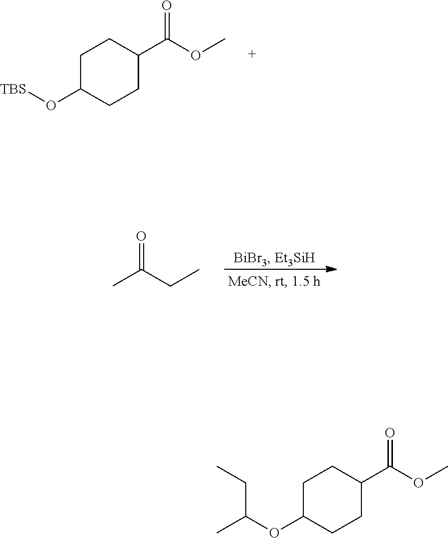

### Step 2: methyl 4-(sec-butoxy)cyclohexane-1-carboxylate

To a solution of methyl 4-((tert-butyldimethylsilyl)oxy)cyclohexane-1-carboxylate (3.3 g, 1 Eq, 12 mmol), bismuth bromide (95 μL, 0.1 Eq, 1.2 mmol) and triethylsilane (2.9 mL, 1.5 Eq, 18 mmol) in acetonitrile (35 mL) was added dropwise methyl ethyl ketone (1.1 mL, 1 Eq, 12 mmol) in Acetonitrile (5.8 mL). The mixture was stirred at room temperature for 1.5 h, the solvent was partially removed, sat. aq. NaHCO₃ was added and the mixture was extracted with EtOAc. Combined organics were washed with brine, dried over Na₂SO₄, filtered and concentrated. Purification by flash column chromatography (SiO₂, 0-100% MTBE in heptane) afforded methyl 4-(sec-butoxy)cyclohexane-1-carboxylate (1.1 g, 5.1 mmol, 42% yield). GCMS: Method 8, 2.61 min, 185.1 [fragment mass]; calcd. 214.31, ¹H NMR (400 MHz, CDCl₃) δ 3.66 (s, 3H), 3.41 (h, J=6.1 Hz, 1H), 3.25 (tt, J=10.6, 3.8 Hz, 1H), 2.26 (tt, J=11.9, 3.5 Hz, 1H), 2.07-1.95 (m, 4H), 1.54-1.21 (m, 6H), 1.11 (d, J=6.2 Hz, 3H), 0.89 (td, J=7.2, 4.3 Hz, 3H).

### Example 6: 1-(4-(sec-butoxy)-2-methylphenyl)ethan-1-one

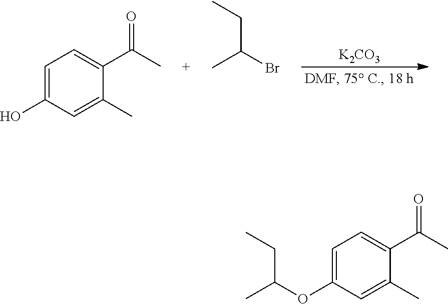

To 1-(4-hydroxy-2-methylphenyl)ethan-1-one (11.0 g, 1 Eq, 73.2 mmol) in DMF (48.8 mL) were added 2-bromobutane (15.1 g, 12.0 mL, 1.5 Eq, 110 mmol) and potassium carbonate (20.2 g, 2 Eq, 146 mmol) and the mixture was heated at 75° C. for 18 h. The reaction was stopped, and the mixture was diluted with water and DCM and layers were separated. The aqueous phase was extracted twice more with DCM and the combined organics were washed with brine, dried over Na₂SO₄, filtered and concentrated. Purification by flash column chromatography (SiO₂, 0-50% MBTE in heptane) afforded 1-(4-(sec-butoxy)-2-methylphenyl)ethan-1-one (11.4 g, 53.5 mmol, 73% yield) as colorless oil. ¹H NMR (400 MHz, CDCl₃) δ 7.77-7.71 (m, 1H), 6.73 (dd, J=6.0, 2.8 Hz, 2H), 4.38 (h, J=6.0 Hz, 1H), 2.56 (s, 3H), 2.54 (s, 3H), 1.81-1.70 (m, 1H), 1.69-1.58 (s, 1H), 1.31 (d, J=6.1 Hz, 3H), 0.98 (t, J=7.5 Hz, 3H).

### Example 7: 2-(4-(sec-butoxy)-2-methylphenyl)propan-2-ol

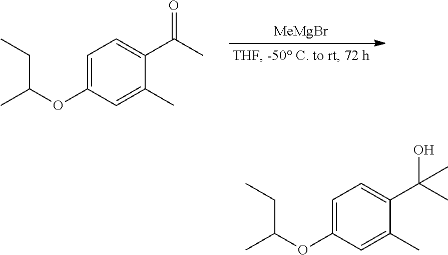 [SMILES: "MeMgBr" ; CCC(C)Oc1ccc(C(C)=O)c(C)c1 ; CCC(C)Oc1ccc(C(C)(C)O)c(C)c1]

To 1-(4-(sec-butoxy)-2-methylphenyl)ethan-1-one (764 mg, 1 Eq, 3.70 mmol) in THF (10 mL) at −50° C. was added dropwise MeMgBr (2.47 mL, 3.0 molar in diethyl ether, 2 Eq, 7.41 mmol) and the mixture was stirred at −50° C. for 4 h. Quenched by addition of sat. aq. NH₄Cl and extracted with EtOAc. The organic phase was dried over Na₂SO₄, filtered and concentrated. Dissolved in THF (10 mL) and cooled to 0° C. and MeMgBr (2.47 mL, 3.0 molar in diethyl ether, 2 Eq, 7.41 mmol) was added dropwise. After addition, the mixture was allowed to warm to room temperature and stirred for 72 h. Quenched by addition of sat. aq. NH₄Cl and extracted with EtOAc. The organic phase was dried over Na₂SO₄, filtered and concentrated. Purification by flash column chromatography (SiO₂, 0-50% MTBE in heptane) afforded 2-(4-(sec-butoxy)-2-methylphenyl)propan-2-ol (427.6 mg, 1.923 mmol, 52% yield). GCMS: Method 8, 2.91 min, 222.0; calcd. 222.3. ¹H NMR (400 MHz, CDCl₃) δ 7.32 (d, J=8.7 Hz, 1H), 6.71 (d, J=2.9 Hz, 1H), 6.65 (dd, J=8.7, 2.8 Hz, 1H), 4.27 (h, J=6.1 Hz, 1H), 2.56 (s, 3H), 1.78-1.60 (m, 8H), 1.28 (d, J=6.1 Hz, 3H), 0.97 (t, J=7.4 Hz, 3H), OH signal not observed.

### Example 8: 1-(4-(sec-butoxy)phenyl)ethan-1-ol

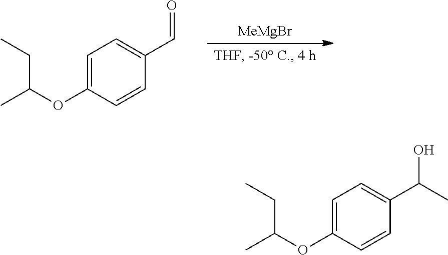

To 4-(sec-butoxy)benzaldehyde (3.00 g, 1 Eq, 16.8 mmol) in THF (60 mL) at −50° C. was added dropwise MeMgBr (11.2 mL, 3.0 molar in diethyl ether, 2 Eq, 33.7 mmol) and the mixture was stirred at −50° C. for 4 h. Quenched by addition of sat. aq. NH₄Cl and extracted with EtOAc. The organic phase was dried over Na₂SO₄, filtered and concentrated. Purification by flash column chromatography (SiO₂, 0-45% EtOAc in heptane) afforded 1-(4-(sec-butoxy)phenyl)ethan-1-ol (1.24 g, 6.38 mmol, 37% yield). LCMS: Method 5, 1.45 min, M+H=177.1 [M−18]; calcd. 195.274, GCMS: Method 8, 2.75 min, 194.0; calcd. 194.2, ¹H NMR (400 MHz, CDCl₃) δ 7.32-7.21 (m, 2H), 6.92-6.82 (m, 2H), 4.85 (qd, J=6.4, 2.5 Hz, 1H), 4.28 (h, J=6.1 Hz, 1H), 1.81-1.55 (m, 2H), 1.47 (s, 3H), 1.29 (dd, J=6.1, 0.9 Hz, 3H), 0.97 (t, J=7.4 Hz, 3H).

### Example 9: 2-(4-(sec-butoxy)phenyl)propan-2-ol

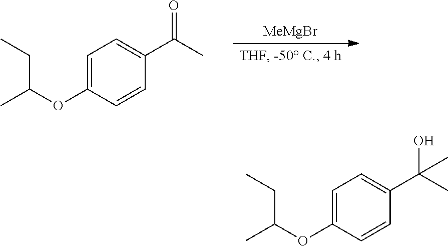

To 1-(4-(sec-butoxy)phenyl)ethan-1-one (3.00 g, 1 Eq, 15.6 mmol) in THF (60 mL) at −50° C. was added dropwise MeMgBr (10.4 mL, 3.0 molar in diethyl ether, 2 Eq, 31.2 mmol) and the mixture was stirred at −50° C. for 4 h. Quenched by addition of sat. aq. NH₄Cl and extracted with EtOAc. The organic phase was dried over Na₂SO₄, filtered and concentrated. Purification by flash column chromatography (SiO₂, 0-50% EtOAc in heptane) afforded 2-(4-(sec-butoxy)phenyl)propan-2-ol (1.84 g, 8.83 mmol, 56% yield). GCMS: Method 8, 2.77 min, 208.1; calcd. 208.3, ¹H NMR (400 MHz, CDCl₃) δ 7.42-7.34 (m, 2H), 6.89-6.81 (m, 2H), 4.28 (h, J=6.0 Hz, 1H), 1.81-1.59 (m, 2H), 1.57 (s, 6H), 1.29 (d, J=6.0 Hz, 3H), 0.97 (t, J=7.5 Hz, 3H), OH signal not observed.

### Example 10: 1-(4-(sec-butoxy)-2-methylphenyl)ethan-1-ol

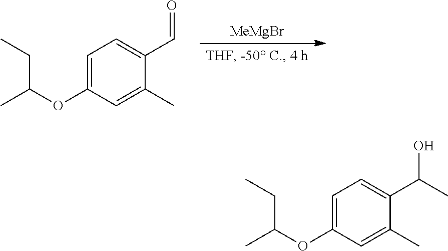

To 4-(sec-butoxy)-2-methylbenzaldehyde (5.00 g, 1 Eq, 26.0 mmol) in THF (75 mL) at −50° C. was added dropwise MeMgBr (17.3 mL, 3.0 molar in diethyl ether, 2 Eq, 52.0 mmol) and the mixture was stirred at −50° C. for 4 h. Quenched by addition of sat. aq. NH₄Cl and extracted with EtOAc. The organic phase was dried over Na₂SO₄, filtered and concentrated. Purification by flash column chromatography (SiO₂, 0-40% EtOAc in heptane) afforded 1-(4-(sec-butoxy)-2-methylphenyl)ethan-1-ol (4.35 g, 20.9 mmol, 80% yield). GCMS: Method 8, 2.87 min, 208.1; calcd. 208.3, ¹H NMR (400 MHz, CDCl₃) δ 7.38 (d, J=8.4 Hz, 1H), 6.75 (dd, J=8.6, 2.7 Hz, 1H), 6.68 (d, J=2.6 Hz, 1H), 5.07 (q, J=6.4 Hz, 1H), 4.27 (h, J=6.0 Hz, 1H), 2.32 (s, 3H), 1.78-1.58 (m, 2H), 1.46 (d, J=6.4 Hz, 3H), 1.28 (dd, J=6.1, 0.9 Hz, 3H), 0.97 (t, J=7.4 Hz, 3H), OH signal not observed.

### Example 11: 1-(4-(sec-butoxy)cyclohexyl)ethan-1-ol

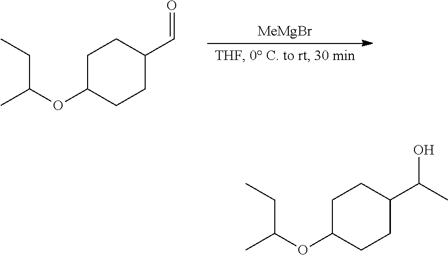

To 4-(sec-butoxy)cyclohexane-1-carbaldehyde (1.35 g, 1 Eq, 7.33 mmol) in THF (14.7 mL) at 0° C. was added methylmagnesium bromide (7.33 mL, 3.0 molar in diethyl ether, 3 Eq, 22.0 mmol). After addition, the mixture was allowed to warm to room temperature and stirred for 30 min. The mixture was quenched by addition of sat. aq. NH₄Cl, concentrated, diluted with NH₄Cl and extracted with DCM. Combined organics were dried over Na₂SO₄, filtered and concentrated. Purification by flash column chromatography (SiO₂, 0-50% MTBE in heptane) and Method I afforded 1-(4-(sec-butoxy)cyclohexyl)ethan-1-ol (593.8 mg, 2.964 mmol, 40% yield) (2.5:25:65:5 mixture of isomers). GCMS: Method 8, 2.53, 2.59, 2.64 and 2.70 min, 171.2 [fragment mass]; calcd. 200.3, ¹H NMR (400 MHz, CDCl₃) δ 3.65-3.13 (m, 3H), 2.09-1.67 (m, 4H), 1.61-0.95 (m, 14H), 0.95-0.81 (m, 3H).

### Example 12: (4-(sec-butoxy)-2-methylphenyl)methanol

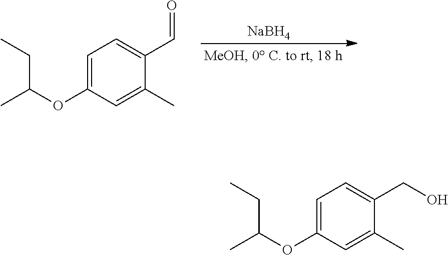

To sodium borohydride (394 mg, 2 Eq, 10.4 mmol) in MeOH (10 mL) at 0° C. was added 4-(sec-butoxy)-2-methylbenzaldehyde (1.00 g, 1 Eq, 5.20 mmol) in MeOH (10 mL). After addition, the mixture was allowed to warm to room temperature and stirred for 18 h. Acidified by addition of 2 M aq. HCl and extracted with diethyl ether. Combined organics were dried over Na₂SO₄, filtered and concentrated. Purification by flash column chromatography (SiO₂, 0-50% EtOAc in heptane), afforded (4-(sec-butoxy)-2-methylphenyl)methanol (665.5 mg, 3.426 mmol, 65% yield). GCMS: Method 8, 2.83 min, 194.1; calcd. 194.2, ¹H NMR (400 MHz, CDCl₃) δ 7.21 (d, J=8.3 Hz, 1H), 6.76-6.66 (m, 2H), 4.63 (s, 2H), 4.29 (h, J=6.0 Hz, 1H), 2.35 (s, 3H), 1.80-1.59 (m, 2H), 1.28 (d, J=6.1 Hz, 3H), 0.97 (t, J=7.5 Hz, 3H), OH signal not observed.

### Example 13: 4-(sec-butoxy)benzyl acetate

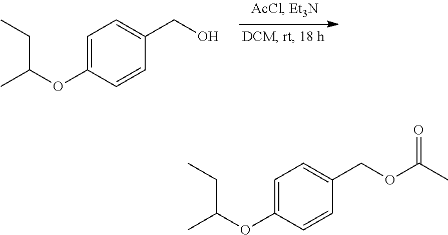 [SMILES: CCC(C)Oc1ccc(CO)cc1 ; CCC(C)Oc1ccc(COC(C)=O)cc1]

To (4-(sec-butoxy)phenyl)methanol (700 mg, 1 Eq, 3.88 mmol) in DCM (10 mL) were added triethylamine (541 μL, 1 Eq, 3.88 mmol) and acetyl chloride (302 μL, 1.1 Eq, 4.27 mmol) and the mixture was stirred at room temperature for 18 h. Purification by flash column chromatography (SiO₂, 0-25% EtOAc in heptane) afforded 4-(sec-butoxy)benzyl acetate (536 mg, 2.41 mmol, 62% yield). GCMS: Method 8, 2.86 min, 222.1; calcd. 222.2, ¹H NMR (400 MHz, CDCl₃) δ 7.28-7.26 (m, 2H), 6.92-6.82 (m, 2H), 5.03 (s, 2H), 4.29 (h, J=6.0 Hz, 1H), 2.08 (s, 3H), 1.81-1.58 (m, 2H), 1.29 (d, J=6.1 Hz, 3H), 0.97 (t, J=7.4 Hz, 3H).

### Example 14: 4-(sec-butoxy)benzyl propionate

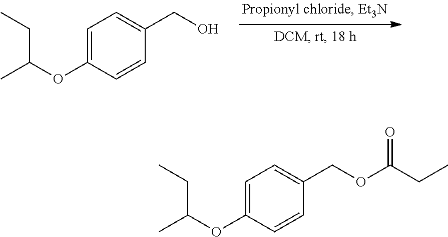 [SMILES: CCC(C)Oc1ccc(CO)cc1 ; CCC(=O)OCc1ccc(OC(C)CC)cc1]

To (4-(sec-butoxy)phenyl)methanol (700 mg, 1 Eq, 3.88 mmol) in DCM (10 mL) were added triethylamine (541 μL, 1 Eq, 3.88 mmol) and propionyl chloride (373 μL, 1.1 Eq, 4.27 mmol) and the mixture was stirred at room temperature for 18 h. Purification by flash column chromatography (SiO₂, 0-25% EtOAc in heptane) afforded 4-(sec-butoxy)benzyl propionate (680.1 mg, 2.878 mmol, 74% yield). GCMS: Method 8, 3.00 min, 236.1; calcd. 236.3, ¹H NMR (400 MHz, CDCl₃) δ 7.28-7.26 (m, 2H), 6.91-6.82 (m, 2H), 5.04 (s, 2H), 4.29 (h, J=6.0 Hz, 1H), 2.35 (q, J=7.5 Hz, 2H), 1.81-1.59 (m, 2H), 1.29 (d, J=6.1 Hz, 3H), 1.15 (s, 3H), 0.96 (s, 3H).

### Example 15: 1-(4-(sec-butoxy)phenyl)ethyl acetate

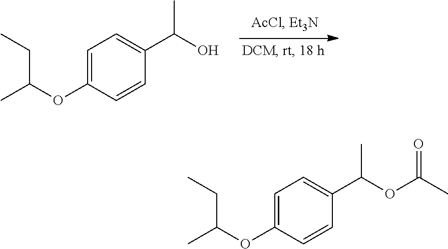 [SMILES: CCC(C)Oc1ccc(C(C)O)cc1 ; CCC(C)Oc1ccc(C(C)OC(C)=O)cc1]

To 1-(4-(sec-butoxy)phenyl)ethan-1-ol (700 mg, 1 Eq, 3.60 mmol) in DCM (10 mL) were added triethylamine (753 μL, 1.5 Eq, 5.40 mmol) and acetyl chloride (382 μL, 1.5 Eq, 5.40 mmol) and the mixture was stirred at room temperature for 18 h. Purification by flash column chromatography (SiO₂, 0-25% EtOAc in heptane) afforded 1-(4-(sec-butoxy)phenyl)ethyl acetate (452.4 mg, 1.914 mmol, 53% yield). GCMS: Method 8, 2.89 min, 236.1; calcd. 236.3, ¹H NMR (400 MHz, CDCl₃) δ 7.28-7.26 (m, 2H), 6.91-6.80 (m, 2H), 5.84 (q, J=6.6 Hz, 1H), 4.28 (h, J=6.0 Hz, 1H), 2.05 (s, 3H), 1.80-1.57 (m, 2H), 1.52 (d, J=6.6 Hz, 3H), 1.28 (d, J=6.1 Hz, 3H), 0.97 (t, J=7.4 Hz, 3H).

### Example 16: 1-(4-(sec-butoxy)phenyl)ethyl propionate

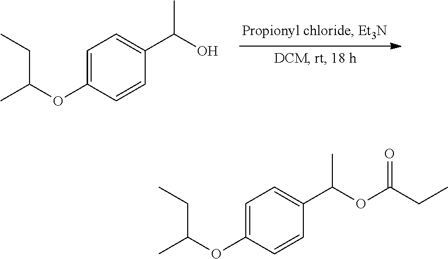 [SMILES: CCC(C)Oc1ccc(C(C)O)cc1 ; CCC(=O)OC(C)c1ccc(OC(C)CC)cc1]

To 1-(4-(sec-butoxy)phenyl)ethan-1-ol (700 mg, 1 Eq, 3.60 mmol) in DCM (10 mL) were added triethylamine (753 μL, 1.5 Eq, 5.40 mmol) and propionyl chloride (471 μL, 1.5 Eq, 5.40 mmol) and the mixture was stirred at room temperature for 18 h. Purification by flash column chromatography (SiO2, 0-25% EtOAc in heptane) afforded 1-(4-(sec-butoxy)phenyl)ethyl propionate (384.7 mg, 1.537 mmol, 42% yield). GCMS: Method 8, 3.01 min, 250.1; calcd. 250.3, ¹H NMR (400 MHz, CDCl₃) δ 7.34-7.17 (m, 2H), 6.91-6.76 (m, 2H), 5.85 (q, J=6.6 Hz, 1H), 4.28 (h, J=6.0 Hz, 1H), 2.32 (qd, J=7.5, 2.7 Hz, 2H), 1.81-1.57 (m, 2H), 1.50 (s, 3H), 1.28 (d, J=6.0 Hz, 3H), 1.12 (t, J=7.6 Hz, 3H), 0.97 (t, J=7.5 Hz, 3H).

### Example 17: 1-(sec-butoxy)-4-(1-methoxyethyl)benzene

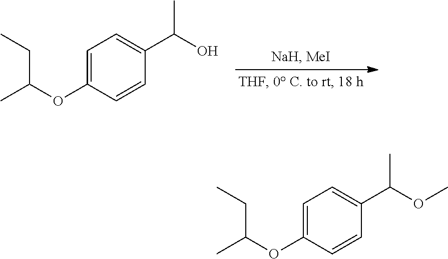

To 1-(4-(sec-butoxy)phenyl)ethan-1-ol (620 mg, 1 Eq, 3.19 mmol) in THF (10 mL) at 0° C. was added sodium hydride (153 mg, 60% Wt in mineral oil, 1.20 Eq, 3.83 mmol) and mixture stirred for 30 min at 0° C. before addition of iodomethane (249 μL, 1.25 Eq, 3.99 mmol). After addition, the mixture was allowed to warm to room temperature and stirred for 18 h. Diluted with water and brine and extracted with DCM (2×). Combined organics were dried over Na₂SO₄, filtered and concentrated. Purification by flash column chromatography (SiO2, 0-25% EtOAc in heptane) afforded 1-(sec-butoxy)-4-(1-methoxyethyl)benzene (476.1 mg, 2.286 mmol, 71% yield). GCMS: Method 8, 2.64 min, 208.1; calcd. 208.3, ¹H NMR (400 MHz, CDCl₃) δ 7.24-7.16 (m, 2H), 6.91-6.79 (m, 2H), 4.31-4.21 (m, 2H), 3.20 (s, 3H), 1.82-1.57 (m, 2H), 1.42 (d, J=6.4 Hz, 3H), 1.29 (dd, J=6.1, 0.9 Hz, 3H), 0.98 (t, J=7.5 Hz, 3H).

### Example 18: 1-(sec-butoxy)-4-(methoxymethyl)benzene

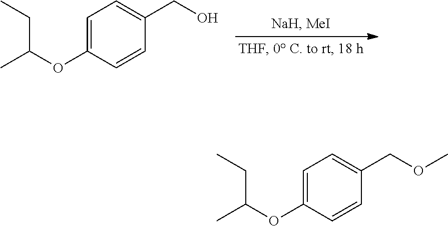

To (4-(sec-butoxy)phenyl)methanol (918 mg, 1 Eq, 5.09 mmol) in THF (20 mL) at 0° C. was added sodium hydride (244 mg, 60% Wt in mineral oil, 1.20 Eq, 6.11 mmol) and the mixture was stirred for 30 min at 0° C. before addition of iodomethane (398 μL, 1.25 Eq, 6.37 mmol). After addition, the mixture was allowed to warm to room temperature and stirred for 18 h. Diluted with water and brine and extracted with DCM (2×). Combined organics were dried over Na₂SO₄, filtered and concentrated. Purification by flash column chromatography (SiO₂, 0-25% EtOAc in heptane) afforded 1-(sec-butoxy)-4-(methoxymethyl)benzene (753.6 mg, 3.879 mmol, 76% yield). GCMS: Method 8, 2.64 min, 194.1; calcd. 194.2, ¹H NMR (400 MHz, CDCl₃) δ 7.25-7.19 (m, 2H), 6.90-6.82 (m, 2H), 4.38 (s, 2H), 4.29 (h, J=6.1 Hz, 1H), 3.36 (s, 3H), 1.81-1.56 (m, 2H), 1.28 (d, J=6.2 Hz, 3H), 0.97 (t, J=7.5 Hz, 3H).

### Example 19: 1-(4-(sec-butoxy)-2-methylphenyl)ethyl acetate

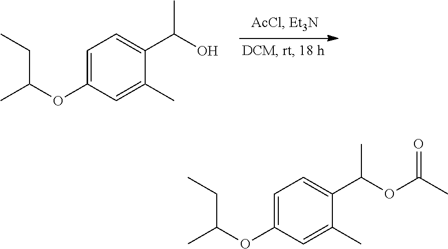 [SMILES: CCC(C)Oc1ccc(C(C)O)c(C)c1 ; CCC(C)Oc1ccc(C(C)OC(C)=O)c(C)c1]

To 1-(4-(sec-butoxy)-2-methylphenyl)ethan-1-ol (1.00 g, 1 Eq, 4.80 mmol) in DCM (10 mL) were added triethylamine (1.34 mL, 2 Eq, 9.60 mmol) and acetyl chloride (508 μL, 1.5 Eq, 7.20 mmol) and the mixture was stirred at room temperature for 18 h. Purification by flash column chromatography (SiO₂, 0-30% EtOAc in heptane) afforded 1-(4-(sec-butoxy)-2-methylphenyl)ethyl acetate (674.6 mg, 2.695 mmol, 56% yield). GCMS: Method 8, 2.97 min, 249.9; calcd. 250.3, ¹H NMR (400 MHz, CDCl₃) δ 7.29 (d, J=8.5 Hz, 1H), 6.73 (dd, J=8.5, 2.7 Hz, 1H), 6.67 (d, J=2.7 Hz, 1H), 6.03 (q, J=6.5 Hz, 1H), 4.27 (h, J=6.1 Hz, 1H), 2.33 (s, 3H), 2.05 (s, 3H), 1.79-1.58 (m, 2H), 1.49 (d, J=6.5 Hz, 3H), 1.28 (d, J=6.1 Hz, 3H), 0.96 (t, J=7.4 Hz, 3H).

### Example 20: 1-(4-(sec-butoxy)-2-methylphenyl)ethyl propionate

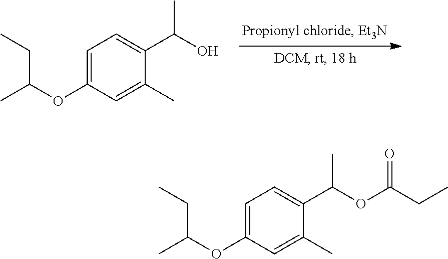 [SMILES: CCC(C)Oc1ccc(C(C)O)c(C)c1 ; CCC(=O)OC(C)c1ccc(OC(C)CC)cc1C]

To 1-(4-(sec-butoxy)-2-methylphenyl)ethan-1-ol (1.00 g, 1 Eq, 4.80 mmol) in DCM (10 mL) were added triethylamine (1.34 mL, 2 Eq, 9.60 mmol) and propionyl chloride (628 μL, 1.5 Eq, 7.20 mmol) and the mixture was stirred at room temperature for 18 h. Purification by flash column chromatography (SiO₂, 0-30% EtOAc in heptane) afforded 1-(4-(sec-butoxy)-2-methylphenyl)ethyl propionate (798.3 mg, 3.020 mmol, 63% yield). GCMS: Method 8, 3.08 min, 264.1; calcd. 264.3, ¹H NMR (400 MHz, CDCl₃) δ 7.28 (d, J=8.5 Hz, 1H), 6.72 (dd, J=8.5, 2.7 Hz, 1H), 6.67 (d, J=2.7 Hz, 1H), 6.03 (q, J=6.6 Hz, 1H), 4.26 (h, J=6.0 Hz, 1H), 2.41-2.24 (m, 5H), 1.79-1.56 (m, 2H), 1.49 (d, 3H), 1.28 (d, J=6.1 Hz, 3H), 1.13 (t, J=7.5 Hz, 3H), 0.96 (t, J=7.4 Hz, 3H).

### Example 21: ethyl (E)-3-(4-(sec-butoxy)phenyl)but-2-enoate

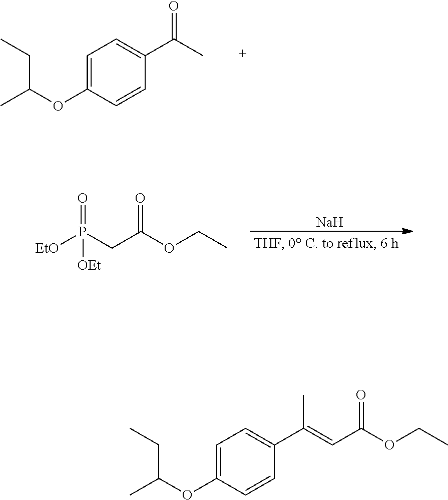

To a suspension of sodium hydride (597 mg, 60% Wt in mineral oil, 1.2 Eq, 14.9 mmol) in THF (10 mL) at 0° C. was added dropwise a solution of diethylphosphonoacetic acid ethyl ester (2.96 mL, 1.2 Eq, 14.9 mmol) in THF (10 mL). The mixture was allowed to warm to room temperature and stirred for 30 min before addition of a solution of 1-(4-(sec-butoxy)phenyl)ethan-1-one (2.39 g, 1 Eq, 12.4 mmol) in THF (10 mL). Stirred at room temperature for 3 h, then heated to reflux and continued for 3 h. The mixture was allowed to cool to room temperature and concentrated. Water and diethyl ether were added and layers separated. The organic phase was dried over Na₂SO₄, filtered and concentrated. Purification by flash column chromatography (SiO₂, 0-50% MTBE in heptane) afforded ethyl (E)-3-(4-(sec-butoxy)phenyl)but-2-enoate (1.89 g, 7.20 mmol, 58% yield). ¹H NMR (400 MHz, CDCl₃) δ 7.47-7.39 (m, 2H), 6.90-6.83 (m, 2H), 6.11 (q, J=1.3 Hz, 1H), 4.33 (h, J=6.0 Hz, 1H), 4.20 (q, J=7.1 Hz, 2H), 2.56 (d, J=1.2 Hz, 3H), 1.82-1.59 (m, 2H), 1.34-1.29 (m, 6H), 0.98 (t, J=7.4 Hz, 3H).

### Example 22: ethyl (E)-3-(4-(sec-butoxy)-2-methylphenyl)-2-methylacrylate

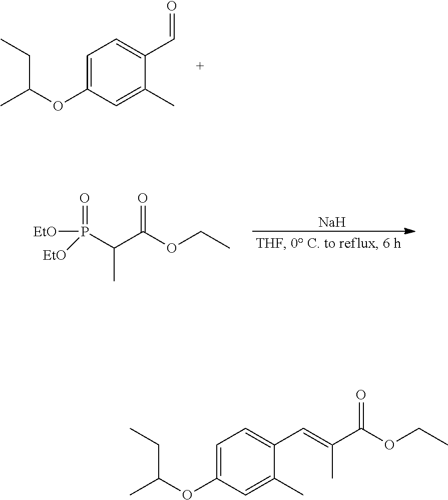

To a suspension of sodium hydride (1.05 g, 60% Wt in mineral oil, 1.2 Eq, 26.2 mmol) in THF (18.2 mL) at 0° C. was added dropwise a solution of ethyl 2-(diethoxyphosphoryl)propanoate (5.63 mL, 1.2 Eq, 26.2 mmol) in THF (18.2 mL). The mixture was allowed to warm to rt and stirred for 30 min before addition of a solution of 4-(sec-butoxy)-2-methylbenzaldehyde (4.20 g, 1 Eq, 21.8 mmol) in THF (18.2 mL). After addition, the mixture was heated under reflux for 30 min. The mixture was allowed to cool to room temperature, diluted with water and diethyl ether and layers separated. The organic phase was dried over Na₂SO₄, filtered and concentrated. Purification by flash column chromatography (SiO₂, 0-50% MTBE in heptane) afforded ethyl (E)-3-(4-(sec-butoxy)-2-methylphenyl)-2-methylacrylate (4.69 g, 17.0 mmol, 78% yield). GCMS: Method 8, 3.34 min, 276.2; calcd. 276.3. ¹H NMR (400 MHz, CDCl₃) δ 7.71 (d, J=1.7 Hz, 1H), 7.16 (d, J=8.3 Hz, 1H), 6.78-6.66 (m, 2H), 4.36-4.18 (m, 3H), 2.27 (s, 3H), 1.99 (d, J=1.5 Hz, 3H), 1.85-1.59 (m, 2H), 1.38-1.28 (m, 6H), 0.98 (t, J=7.4 Hz, 3H).

### Example 23: Ethyl (E)-3-(4-(sec-butoxy)phenyl)-2-

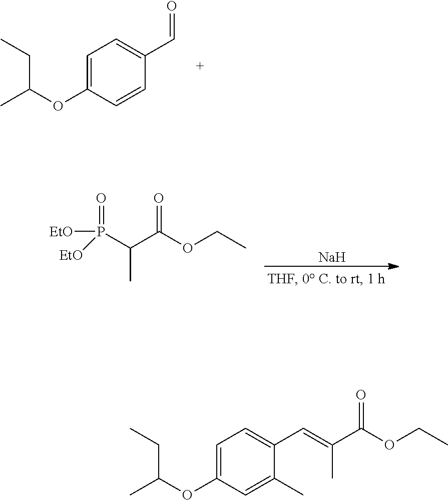

To a solution of sodium hydride (2.02 g, 60% Wt, 1.2 Eq, 50.5 mmol) in THF (25 mL) at 0° C. was added dropwise a solution of ethyl 2-(diethoxyphosphoryl)propanoate (12.0 g, 10.8 mL, 1.2 Eq, 50.5 mmol) in THF (25 mL). The mixture was allowed to warm to room temperature and stirred for 30 min before addition of a solution of 4-(sec-butoxy)benzaldehyde (7.50 g, 1 Eq, 42.1 mmol) in THF (25 mL). The resulting reacting mixture was stirred at rt for 1 h. The reaction was stopped, water and diethyl ether were added and the layers were separated. The organic layer was washed one time more with water and then with brine. The organic phase was dried over Na₂SO₄, filtered and concentrated. The crude was purified by flash column chromatography (SiO₂, 0-50% MBTE in heptane) to afford ethyl (E)-3-(4-(sec-butoxy)phenyl)-2-methylacrylate (10.05 g, 35 mmol, 84% yield) as colorless oil. ¹H NMR (400 MHz, CDCl₃) δ 7.63 (d, J=1.7 Hz, 1H), 7.39-7.35 (m, 2H), 6.93-6.87 (m, 2H), 4.34 (h, J=6.1 Hz, 1H), 4.26 (q, J=7.1 Hz, 2H), 2.13 (d, J=1.4 Hz, 3H), 1.82-1.70 (m, 1H), 1.69-1.58 (m, 1H), 1.37-1.30 (m, 6H), 0.98 (t, J=7.5 Hz, 3H).

### Example 24: ethyl (E)-3-(4-(sec-butoxy)cyclohexyl)acrylate

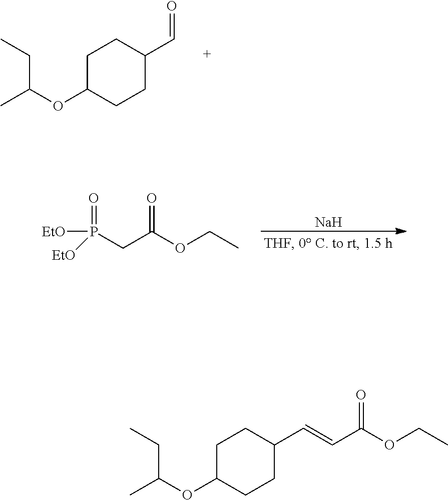

To sodium hydride (391 mg, 60% Wt in mineral oil, 1.2 Eq, 9.77 mmol) in THF (5.43 mL) at 0° C. was added dropwise a solution of diethylphosphonoacetic acid ethyl ester (1.94 mL, 1.2 Eq, 9.77 mmol) in THF (5.43 mL). The mixture was allowed to warm to room temperature and stirred for 30 min before addition of a solution of 4-(sec-butoxy)cyclohexane-1-carbaldehyde (1.50 g, 1 Eq, 8.14 mmol) in THF (5.43 mL). The mixture was stirred at room temperature for 1 h, water and diethyl ether were added and layers separated. The organic phase was dried over Na₂SO₄, filtered and concentrated. Purification by flash column chromatography (SiO₂, 0-40% MTBE in heptane) afforded ethyl (E)-3-(4-(sec-butoxy)cyclohexyl)acrylate (1.25 g, 4.91 mmol, 60% yield). ¹H NMR (400 MHz, CDCl₃) δ 6.92 (ddd, J=25.2, 15.8, 6.7 Hz, 1H), 5.78 (ddd, J=15.9, 4.4, 1.5 Hz, 1H), 4.18 (q, J=7.1 Hz, 2H), 3.45-3.32 (m, 1H), 3.22 (tt, J=10.5, 4.1 Hz, 1H), 2.26-1.96 (m, 2H), 1.84-1.75 (m, 2H), 1.55-1.36 (m, 3H), 1.31-1.24 (m, 4H), 1.24-1.15 (m, 1H), 1.10 (dd, J=7.7, 6.1 Hz, 3H), 0.93-0.85 (m, 5H).

### Example 25: ethyl (E)-3-(4-(sec-butoxy)-2-methylphenyl)but-2-enoate

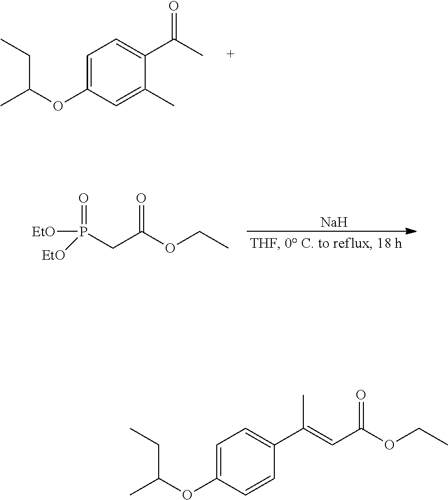

To sodium hydride (0.93 g, 60% Wt in mineral oil, 1.2 Eq, 23 mmol) in THF (13 mL) at 0° C. was added dropwise a solution of diethylphosphonoacetic acid ethyl ester (4.6 mL, 1.2 Eq, 23 mmol) in THF (13 mL). The mixture was allowed to warm to room temperature and stirred for 30 min before addition of a solution of 1-(4-(sec-butoxy)-2-methylphenyl)ethan-1-one (4.0 g, 1 Eq, 19 mmol) in THF (13 mL). The mixture was stirred at room temperature for 3 h, then heated under reflux for 3 h and then allowed to cool to room temperature and stirred for 12 h. Water and diethyl ether were added, layers separated, and organic phase was dried over Na₂SO₄, filtered and concentrated. Purification by flash column chromatography (SiO₂, 0-50% MTBE in heptane) afforded ethyl (E)-3-(4-(sec-butoxy)-2-methylphenyl)but-2-enoate (3.36 g, 12.2 mmol, 63% yield). GCMS: Method 8, 3.33 min, 276.1; calcd. 276.3.

### Example 26: Ethyl 3-(4-(sec-butoxy)phenyl)-2-methylbut-2-enoate

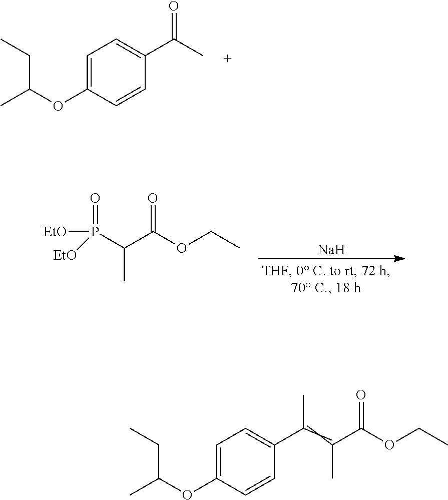

To a solution of sodium hydride (1.50 g, 60% Wt, 1.2 Eq, 37.5 mmol) in THF (20.8 mL) at 0° C. was added dropwise a solution of Triethyl-2-Phosphonopropionate (8.94 g, 8.05 mL, 1.2 Eq, 37.5 mmol) in THF (20.8 mL). The mixture was allowed to warm to room temperature and stirred for 30 min before addition of a solution of 1-(4-(sec-butoxy)phenyl)ethan-1-one (6.01 g, 1 Eq, 31.3 mmol) in THF (20.8 mL). The reaction mixture was stirred at room temperature for 72 h and at 70° C. for 18 h. The reaction was stopped, water and diethyl ether were added and the layers were separated. The organic layer was washed one time more with water and then with brine. The organic phase was dried over Na₂SO₄, filtered and concentrated. The crude was purified by flash column chromatography (SiO₂, 0-15% EtOAc in heptane) to afford ethyl 3-(4-(sec-butoxy)phenyl)-2-methylbut-2-enoate (5.21 g, 17 mmol, 55% yield) as colorless oil (60:40 mixture of isomers). LCMS: Method 10, 2.31 and 2.39 min, M+H=277.1; calcd. 277.172. ¹H NMR (400 MHz, CDCl₃) δ 7.10-7.01 (m, 2H), 6.89-6.84 (m, 2H), 4.34-4.22 (m, 3H), 2.24 (q, J=1.5 Hz, 3H), 1.79 (q, J=1.6 Hz, 3H), 1.78-1.69 (m, 1H), 1.65-1.55 (m, 1H), 1.36-1.29 (m, 3H), 0.99 (t, J=7.3 Hz, 3H), 0.88 (td, J=7.1, 1.4 Hz, 3H).

### Example 27: methyl 2-amino-4-(sec-butoxy)benzoate

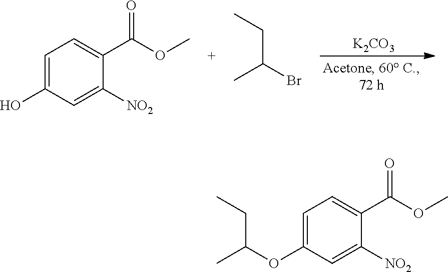

### Step 1: methyl 4-(sec-butoxy)-2-nitrobenzoate

To methyl 4-hydroxy-2-nitrobenzoate (755 mg, 1 Eq, 3.83 mmol) in acetone (10 mL) were added potassium carbonate (794 mg, 1.5 Eq, 5.74 mmol) and 2-bromobutane (502 μL, 1.2 Eq, 4.60 mmol) and the mixture was heated at 60° C. for 3 h. Potassium carbonate (794 mg, 1.5 Eq, 5.74 mmol) and 2-bromobutane (502 μL, 1.2 Eq, 4.60 mmol) were added and the mixture was heated at 60° C. for 4 h. 2-bromobutane (502 μL, 1.2 Eq, 4.60 mmol) was added and the mixture was heated at 60° C. for 5 h. Potassium carbonate (794 mg, 1.5 Eq, 5.74 mmol) and 2-bromobutane (502 μL, 1.2 Eq, 4.60 mmol) were added the mixture was heated at 60° C. for 60 h. The mixture was concentrated, partitioned between water and DCM and the aqueous phase was extracted with DCM. Combined organics were washed with brine, dried over Na₂SO₄, filtered and concentrated. Purification by flash column chromatography (SiO₂, 0-45% EtOAc in heptane) afforded methyl 4-(sec-butoxy)-2-nitrobenzoate (792 mg, 3.13 mmol, 81% yield). ¹H NMR (400 MHz, CDCl₃) δ 7.77 (d, J=8.7 Hz, 1H), 7.19 (d, J=2.5 Hz, 1H), 7.06 (dd, J=8.7, 2.5 Hz, 1H), 4.40 (h, J=6.1 Hz, 1H), 3.87 (s, 3H), 1.84-1.60 (m, 2H), 1.33 (d, J=6.1 Hz, 3H), 0.98 (t, J=7.5 Hz, 3H).

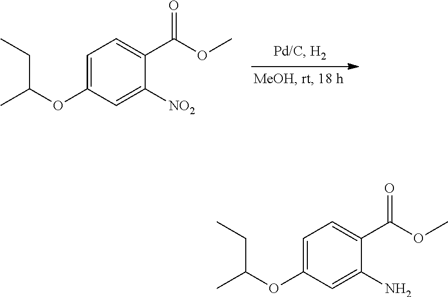 [SMILES: CCC(C)Oc1ccc(C(=O)OC)c([N+](=O)[O-])c1 ; CCC(C)Oc1ccc(C(=O)OC)c(N)c1]

### Step 2: methyl 2-amino-4-(sec-butoxy)benzoate

To methyl 4-(sec-butoxy)-2-nitrobenzoate (792 mg, 1 Eq, 3.13 mmol) in MeOH (20 mL) under nitrogen atmosphere was added Pd/C (333 mg, 5% Wt, 0.05 Eq, 156 mol). The mixture was placed under hydrogen atmosphere via 3 vacuum hydrogen cycles and stirred at room temperature for 18 h. The mixture was filtered over celite and the filter cake was rinsed with MeOH. The filtrate was concentrated, affording methyl 2-amino-4-(sec-butoxy)benzoate (635 mg, 2.84 mmol, 91% yield). LCMS: Method 2, 1.82 min, M+H=224.1; calcd. 224.272, GCMS: Method 8, 3.17 min, 223.1; calcd. 223.2, ¹H NMR (400 MHz, CDCl₃) δ 7.77 (d, J=9.0 Hz, 1H), 6.21 (dd, J=8.9, 2.4 Hz, 1H), 6.09 (d, J=2.4 Hz, 1H), 5.73 (brs, 2H), 4.30 (h, J=6.1 Hz, 1H), 3.83 (s, 3H), 1.80-1.57 (m, 2H), 1.29 (d, J=6.1 Hz, 3H), 0.96 (t, J=7.5 Hz, 3H).

### Example 28: 4-(sec-butoxy)-1-isobutyl-2-methylbenzene

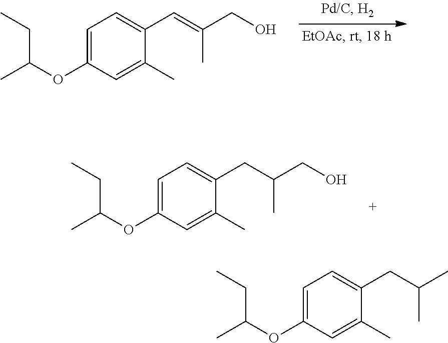 [SMILES: CCC(C)Oc1ccc(/C=C(\C)CO)c(C)c1 ; CCC(C)Oc1ccc(CC(C)CO)c(C)c1 ; CCC(C)Oc1ccc(CC(C)C)c(C)c1]

To 3-(4-(sec-butoxy)-2-methylphenyl)-2-methylprop-2-en-1-ol (3.73 g, 1 Eq, 14.8 mmol) in EtOAc (74.0 mL) under nitrogen atmosphere was added Pd/C (1.58 g, 5% Wt, 0.05 Eq, 740 mol). The mixture was placed under hydrogen atmosphere via 3 vacuum hydrogen cycles and stirred at room temperature for 18 h. The mixture was filtered over celite, the filter cake was rinsed with MeOH, and the filtrate was concentrated. Purification by flash column chromatography (SiO₂, 0-50% MTBE in heptane) afforded 3-(4-(sec-butoxy)-2-methylphenyl)-2-methylpropan-1-ol (1.62 g, 6.85 mmol, 46% yield), LCMS: Method 9, 2.02 min, M+H=237.2; calcd. 237.37, ¹H NMR (400 MHz, CDCl₃) δ 6.99 (d, J=8.3 Hz, 1H), 6.70 (d, J=2.7 Hz, 1H), 6.65 (dd, J=8.3, 2.7 Hz, 1H), 4.24 (h, J=6.0 Hz, 1H), 3.60-3.43 (m, 2H), 2.67 (dd, J=13.8, 6.6 Hz, 1H), 2.34 (dd, J=13.8, 7.9 Hz, 1H), 2.27 (s, 3H), 1.89 (ddt, J=12.5, 8.0, 6.3 Hz, 1H), 1.74-1.58 (m, 2H), 1.27 (d, J=6.1 Hz, 3H), 0.99-0.91 (m, 6H) OH signal not observed; and 4-(sec-butoxy)-1-isobutyl-2-methylbenzene (1415.2 mg, 6.4222 mmol, 43% yield), LCMS: Method 4, 4.13 min, M+H=221.0; calcd. 221.356, GCMS: Method 8, 2.77 min, 220.2; calcd. 220.3, ¹H NMR (400 MHz, CDCl₃) δ 6.96 (d, J=8.3 Hz, 1H), 6.71-6.60 (m, 2H), 4.24 (h, J=6.1 Hz, 1H), 2.40 (d, J=7.2 Hz, 2H), 2.25 (s, 3H), 1.88-1.66 (m, 2H), 1.66-1.55 (m, 1H), 1.27 (d, J=6.1 Hz, 3H), 0.97 (t, J=7.5 Hz, 3H), 0.91 (d, J=6.6 Hz, 6H).

### Example 29: 3-(4-(sec-butoxy)phenyl)-2-methylpropan-1-ol

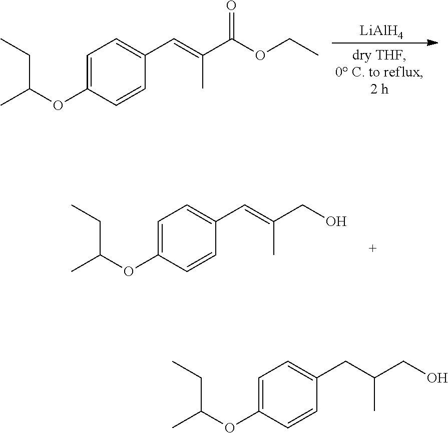

### Step 1: Mixture of (E)-3-(4-(sec-butoxy)phenyl)-2-methylprop-2-en-1-ol and 3-(4-(sec-butoxy)phenyl)-2-methylpropan-1-ol

To lithium aluminum hydride (1.52 g, 16.7 mL, 2.4 molar in THF, 3 Eq, 40.0 mmol) at 0° C. under nitrogen atmosphere was added a solution of ethyl (E)-3-(4-(sec-butoxy)phenyl)-2-methylacrylate (3.50 g, 1 Eq, 13.3 mmol) in THF (25 mL) and after addition, the mixture was allowed to warm to room temperature and then stirred under reflux for 2 h. The reaction was cooled on ice and dropwise sodium potassium tartrate solution (88.9 mL, 1.5 molar, 10 Eq, 133 mmol) was added until gas formation ceased. The mixture was extracted using EtOAc, combined organics were washed with water, brine, dried over Na₂SO₄, filtered and concentrated to give (E)-3-(4-(sec-butoxy)phenyl)-2-methylprop-2-en-1-ol and 3-(4-(sec-butoxy)phenyl)-2-methylpropan-1-ol (2.78 g, 6.28 mmol, 94% yield) (67:33 mixture). GCMS: Method 8, 3.15 min, 220.1; calcd. 220.3, 3.03 min, 222.1; calcd. 222.3.

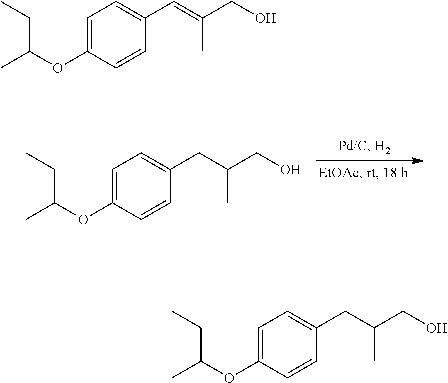 [SMILES: CCC(C)Oc1ccc(/C=C(\C)CO)cc1 ; CCC(C)Oc1ccc(CC(C)CO)cc1 ; CCC(C)Oc1ccc(CC(C)CO)cc1]

### Step 2: 3-(4-(sec-butoxy)phenyl)-2-methylpropan-1-ol

To a mixture of (E)-3-(4-(sec-butoxy)phenyl)-2-methylprop-2-en-1-ol and 3-(4-(sec-butoxy)phenyl)-2-methylpropan-1-ol (2.78 g, 1 Eq, 6.28 mmol) in EtOAc (50 mL) under nitrogen atmosphere was added Pd/C (668 mg, 5% Wt, 0.05 Eq, 314 mol). The mixture was placed under hydrogen atmosphere via 3 vacuum hydrogen cycles and stirred at room temperature for 18 h. The mixture was filtered over celite, the filter cake was rinsed with EtOAc, and the filtrate was concentrated. Purification by flash column chromatography (SiO₂, 0-50% MTBE in heptane) afforded 3-(4-(sec-butoxy)phenyl)-2-methylpropan-1-ol (1.63 g, 7.33 mmol, 58% yield). GCMS: Method 8, 3.05 min, 222.1; calcd. 222.3, ¹H NMR (400 MHz, CDCl₃) δ 7.10-7.02 (m, 2H), 6.87-6.76 (m, 2H), 4.25 (h, J=6.0 Hz, 1H), 3.57-3.41 (m, 2H), 2.67 (dd, J=13.6, 6.4 Hz, 1H), 2.37 (dd, J=13.6, 7.9 Hz, 1H), 1.90 (dp, J=7.9, 6.3 Hz, 1H), 1.74 (tt, J=13.6, 7.5 Hz, 1H), 1.65-1.54 (m, 2H), 1.28 (d, J=6.1 Hz, 3H), 0.97 (t, J=7.4 Hz, 3H), 0.91 (d, J=6.8 Hz, 3H), OH signal not observed.

### Example 30: ethyl 3-(4-(sec-butoxy)cyclohexyl)propanoate

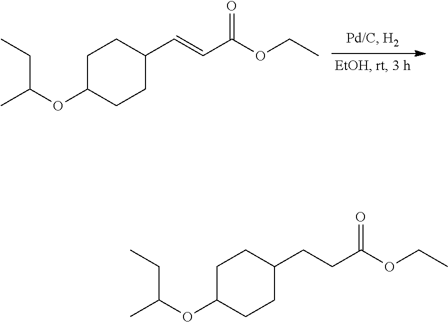

To ethyl 3-(4-(sec-butoxy)cyclohexyl)acrylate (1.25 g, 1 Eq, 4.91 mmol) in Ethanol (24.6 mL) under nitrogen atmosphere was added Pd/C (523 mg, 5% Wt, 0.05 Eq, 246 mol). The mixture was placed under hydrogen atmosphere via 3 vacuum hydrogen cycles and stirred at room temperature for 3 h. The mixture was filtered over celite, the filter cake was rinsed with EtOH and EtOAc, and the filtrate was concentrated. Purification by flash column chromatography (SiO₂, 0-50% MTBE in heptane) afforded ethyl 3-(4-(sec-butoxy)cyclohexyl)propanoate (1.0 g, 3.9 mmol, 79% yield) (33:67 mixture of isomers). GCMS: Method 8, 2.98 and 3.02 min, 227.1 [fragment mass]; calcd. 256.4, ¹H NMR (400 MHz, CDCl₃) δ 4.12 (q, J=7.2 Hz, 2H), 3.59-3.52 (m, 0.3H), 3.45-3.37 (m, 0.7H), 3.37-3.29 (m, 0.3H), 3.25-3.14 (m, 0.7H), 2.34-2.25 (m, 2H), 2.02-1.90 (m, 1H), 1.82-1.66 (m, 2H), 1.57-1.30 (m, 6H), 1.30-1.13 (m, 6H), 1.09 (t, J=5.9 Hz, 3H), 1.01-0.83 (m, 4H).

### Example 31: ethyl 3-(4-(sec-butoxy)-2-methylphenyl)butanoate

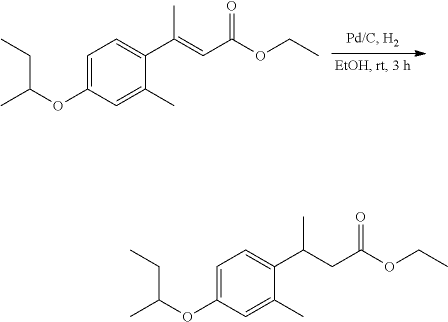

To ethyl 3-(4-(sec-butoxy)-2-methylphenyl)but-2-enoate (3.36 g, 1 Eq, 12.2 mmol) in Ethanol (60.8 mL) under nitrogen atmosphere was added Pd/C (1.29 g, 5% Wt, 0.05 Eq, 608 mol). The mixture was placed under hydrogen atmosphere via 3 vacuum hydrogen cycles and stirred at room temperature for 3 h. The mixture was filtered over celite, the filter cake was rinsed with EtOH and EtOAc, and the filtrate was concentrated, affording ethyl 3-(4-(sec-butoxy)-2-methylphenyl)butanoate (3.3 g, 11 mmol, 94% yield), of which 300 mg was purified by flash column chromatography (SiO₂, 0-50% MTBE in heptane), affording ethyl 3-(4-(sec-butoxy)-2-methylphenyl)butanoate (246.6 mg, 885.8 mol). GCMS: Method 8, 3.23 min, 278.2; calcd. 278.3, ¹H NMR (400 MHz, CDCl₃) δ 7.06 (d, J=8.2 Hz, 1H), 6.72-6.65 (m, 2H), 4.24 (h, J=6.1 Hz, 1H), 4.08 (q, J=7.2 Hz, 2H), 3.51-3.39 (m, 1H), 2.62-2.43 (m, 2H), 2.33 (s, 3H), 1.79-1.66 (m, 1H), 1.65-1.57 (m, 1H), 1.27 (d, J=6.1 Hz, 3H), 1.23 (d, J=6.9 Hz, 3H), 1.18 (t, J=7.1 Hz, 3H), 0.96 (t, J=7.4 Hz, 3H).

### Example 32: Ethyl 3-(4-(sec-butoxy)cyclohexyl)-2-methylpropanoate

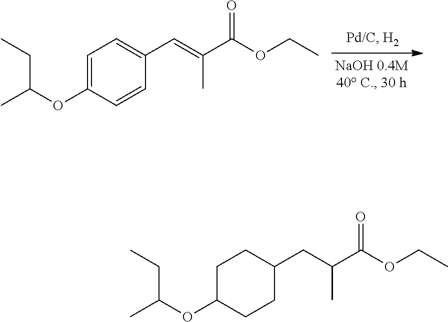

A suspension of ethyl (E)-3-(4-(sec-butoxy)phenyl)-2-methylacrylate (3.97 g, 1 Eq, 15.1 mmol), Rhodium, 5% on activated carbon (405 mg, 5% Wt, 0.013 Eq, 197 mol) in NaOH aq. (1.82 g, 113 mL, 0.4 molar, 3 Eq, 45.4 mmol) was put under 30 bar hydrogen (520 mg, 6.20 L, 17 Eq, 257 mmol) (3× flushing) atmosphere and stirred with a mechanical top-stirrer at 200 rpm at 40° C. for 30 h. After reaction completion, the vessel was purged with nitrogen and the mixture was diluted with ethanol, filtered through a layer of cotton, rinsed with ethanol and water, and concentrated to remove most of the organic solvents. The water layer was acidified using KHSO₄ to a pH ˜1, then extracted with DCM (3×). The combined organic layers were dried with Na₂SO₄, filtered and concentrated, to give ethyl 3-(4-(sec-butoxy)cyclohexyl)-2-methylpropanoate (2.94 g, 10 mmol, 68% yield) as a colorless oil. GCMS: Method 8, 3.00 min, 270.2; calcd. 270.219.

### Example 33: Ethyl 3-(4-(sec-butoxy)phenyl)-2-methylbutanoate

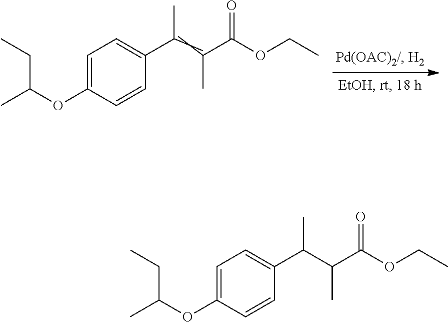

To ethyl 3-(4-(sec-butoxy)phenyl)-2-methylbut-2-enoate (5.21 g, 1 Eq, 18.9 mmol) in EtOH (60 mL) under nitrogen atmosphere was added Pd(OAc)₂/C (750 mg, 5% Wt, 0.0187 Eq, 352 mol). The mixture was placed under hydrogen atmosphere via 3 vacuum hydrogen cycles and stirred at rt for 18 h. After reaction completion, the mixture was filtered through a thin pad of celite, and the residue was rinsed twice with EtOH. The filtrate was concentrated, affording ethyl 3-(4-(sec-butoxy)phenyl)-2-methylbutanoate (4.53 g, 16.3 mmol, 86% yield) as a colorless oil (58:42 mixture of isomers). LCMS: Method 10, 2.35 and 2.37 min, M+H=279.1; calcd. 279.188. GCMS: Method 8, 3.17 and 3.20 min, 121.0 [main signal-fragment]; calcd. 278.188.

### Example 34: Ethyl 3-(4-(sec-butoxy)cyclohexyl)butanoate

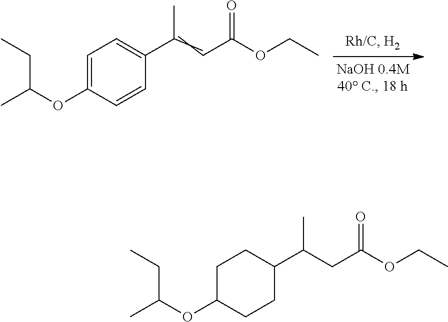

A suspension of ethyl 3-(4-(sec-butoxy)phenyl)but-2-enoate (5.10 g, 1 Eq, 19.4 mmol), rhodium, 5% on activated carbon (520 mg, 5% Wt, 0.013 Eq, 253 mol) in NaOH aq. (2.33 g, 146 mL, 0.4 molar, 3 Eq, 58.3 mmol) was put under 30 bar hydrogen atmosphere (668 mg, 7.97 L, 17 Eq, 330 mmol) (3× flushing) and stirred with a mechanical top-stirrer at 200 rpm at 40° C. After reaction completion, the vessel was purged with nitrogen and the mixture was diluted with ethanol, filtered through a layer of cotton, rinsed with ethanol and water, and concentrated to remove most of the organic solvents. The water layer was acidified using KHSO₄ to a pH ˜1, then extracted with DCM (3×). The combined organic layers were dried with Na₂SO₄, filtered and concentrated, to give ethyl 3-(4-(sec-butoxy)cyclohexyl)butanoate (3.03 g, 11 mmol, 56% yield) as a colorless oil (82:18 mixture of isomers). GCMS: Method 8, 3.06 min and 3.13 min, 197.1 [main signal-fragment]; calcd. 270.219.

### Example 35: Ethyl 3-(4-(sec-butoxy)-2-methylcyclohexyl)butanoate

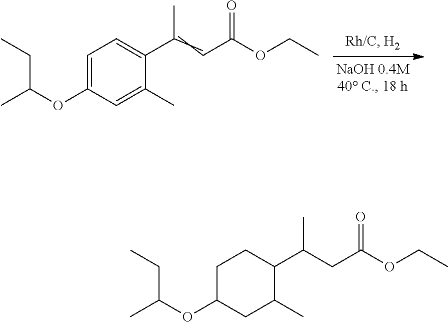

A suspension of ethyl 3-(4-(sec-butoxy)-2-methylphenyl)but-2-enoate (4.05 g, 1 Eq, 14.7 mmol), rhodium, 5% on activated carbon (392 mg, 5% Wt, 0.013 Eq, 190 mol) in NaOH aq. (1.76 g, 110 mL, 0.4 molar, 3 Eq, 44.0 mmol) was put under 30 bar hydrogen atmosphere (503 mg, 6.01 L, 17 Eq, 249 mmol) (3× flushing) and stirred with a mechanical top-stirrer at 200 rpm at 40° C. After reaction completion, the vessel was purged with nitrogen and the mixture was then diluted with ethanol and filtered through a layer of cotton. The filter cake was then rinsed with water, ethanol and THF, then the filtrate was concentrated to remove most of the organic solvents. The resulting water layer was acidified using KHSO₄ to a pH ˜1, then the organics were extracted with DCM (3×). The combined organic layers were dried with Na₂SO₄, filtered and concentrated. The crude mixture was further purified by flash column chromatography (SiO₂, 0-20% MBTE in heptane), to give ethyl 3-(4-(sec-butoxy)-2-methylcyclohexyl)butanoate (1.37 g, 4.82 mmol, 33% yield) as a colorless oil (mixture of isomers). ¹H NMR (400 MHz, CDCl₃) δ 4.12 (q, J=7.1 Hz, 2H), 3.55 (ddd, J=11.7, 7.3, 3.8 Hz, 1H), 3.36 (ddt, J=10.1, 8.0, 3.1 Hz, 1H), 2.56-2.38 (m, 1H), 2.05-1.82 (m, 3H), 1.82-1.63 (m, 1H), 1.59-1.29 (m, 6H), 1.25-1.17 (m, 4H), 1.09-0.96 (m, 6H), 0.97-0.80 (m, 7H).

### Example 36: 3-(4-(sec-butoxy)phenyl)propan-1-ol

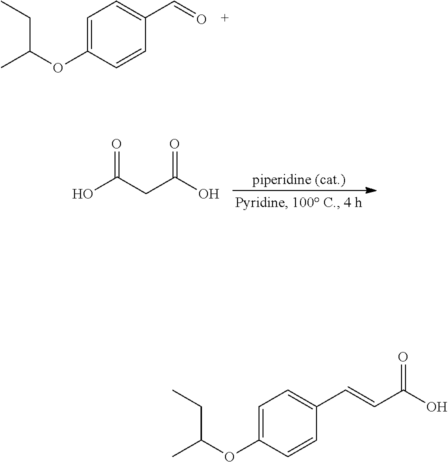 [SMILES: CCC(C)Oc1ccc(C=O)cc1 ; O=C(O)CC(=O)O ; "piperidine (cat.)" ; CCC(C)Oc1ccc(/C=C/C(=O)O)cc1]

### Step 1: (E)-3-(4-(sec-butoxy)phenyl)acrylic acid

To 4-(sec-butoxy)benzaldehyde (8.00 g, 1 Eq, 44.9 mmol) in pyridine (100 mL) were added malonic acid (5.77 mL, 2 Eq, 89.8 mmol) and piperidine (443 μL, 0.1 Eq, 4.49 mmol) and the mixture was heated at 100° C. for 4 h. Allowed to cool to room temperature, acidified by addition of 2M aq. HCl, extracted with DCM, dried over Na₂SO₄, filtered and concentrated, which afforded (E)-3-(4-(sec-butoxy)phenyl)acrylic acid (9.42 g, 42.8 mmol, 95% yield). LCMS: Method 9, 1.91 min, M+H=221.2; calcd. 221.27, ¹H NMR (400 MHz, DMSO) δ 12.20 (brs, 1H), 7.64-7.58 (m, 2H), 7.53 (d, J=15.9 Hz, 1H), 6.96-6.90 (m, 2H), 6.35 (d, J=16.0 Hz, 1H), 4.46 (h, J=6.0 Hz, 1H), 1.74-1.51 (m, 2H), 1.23 (d, J=6.0 Hz, 3H), 0.92 (t, J=7.4 Hz, 3H).

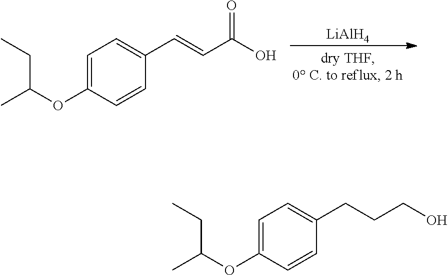 [SMILES: CCC(C)Oc1ccc(/C=C/C(=O)O)cc1 ; "LiAlH4" ; CCC(C)Oc1ccc(CCCO)cc1]

### Step 2: 3-(4-(sec-butoxy)phenyl)propan-1-ol

To lithium aluminum hydride (17.9 mL, 2.4 molar in THF, 3 Eq, 42.9 mmol) at 0° C. under nitrogen atmosphere was added a solution of (E)-3-(4-(sec-butoxy)phenyl)acrylic acid (3.15 g, 1 Eq, 14.3 mmol) in anhydrous THF (20 mL) and after addition, the mixture was allowed to warm to room temperature and then stirred under reflux for 2 h. The mixture was allowed to cool to room temperature, cooled on ice and portion wise Na₂SO₄·10H₂O was added until gas formation ceased. The suspension was filtered over celite, the filter cake was rinsed with hot THF, and the filtrate was concentrated. Purification by flash column chromatography (SiO₂, 0-60% EtOAc in heptane) afforded 3-(4-(sec-butoxy)phenyl)propan-1-ol (1.71 g, 8.21 mmol, 57% yield). GCMS: Method 8, 2.97 min, 208.1; calcd. 208.3, ¹H NMR (400 MHz, CDCl₃) δ 7.13-7.04 (m, 2H), 6.85-6.77 (m, 2H), 4.26 (hept, J=6.0 Hz, 1H), 3.67 (td, J=6.4, 5.2 Hz, 2H), 2.64 (dd, J=8.6, 6.7 Hz, 2H), 1.92-1.81 (m, 2H), 1.80-1.55 (m, 2H), 1.28 (d, J=6.1 Hz, 3H), 0.97 (t, J=7.5 Hz, 3H), OH signal not observed.

### Example 37: 3-(4-(sec-butoxy)phenyl)butan-1-ol

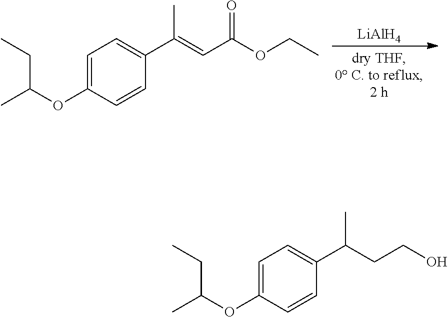

To lithium aluminum hydride (9.01 mL, 2.4 molar in THF, 3 Eq, 21.6 mmol) at 0° C. under nitrogen atmosphere was added a solution of ethyl (E)-3-(4-(sec-butoxy)phenyl)but-2-enoate (1.89 g, 1 Eq, 7.20 mmol) in anhydrous THF (10 mL) and after addition, the mixture was allowed to warm to room temperature and then stirred under reflux for 2 h. The mixture was allowed to cool to room temperature, cooled on ice, and portion wise Na₂SO₄·10H₂O was added until gas formation ceased. The suspension was filtered over celite, the filter cake was rinsed with hot THF, and the filtrate was concentrated. Purification by flash column chromatography (SiO₂, 0-60% EtOAc in heptane) afforded 3-(4-(sec-butoxy)phenyl)butan-1-ol (1.07 g, 4.81 mmol, 67% yield). GCMS: Method 8, 3.01 min, 222.1; calcd. 222.3, ¹H NMR (400 MHz, CDCl₃) δ 7.13-7.06 (m, 2H), 6.85-6.79 (m, 2H), 4.25 (h, J=6.0 Hz, 1H), 3.63-3.49 (m, 2H), 2.89-2.77 (m, 1H), 1.90-1.58 (m, 4H), 1.26 (dd, J=13.4, 6.5 Hz, 6H), 0.97 (t, J=7.5 Hz, 3H), OH signal not observed.

### Example 38: 3-(4-(sec-butoxy)-2-methylphenyl)-2-methylprop-2-en-1-ol

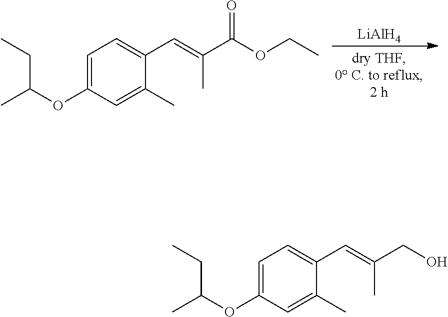

To lithium aluminum hydride (21.0 mL, 2.4 molar in THF, 3 Eq, 50.5 mmol) at 0° C. under nitrogen atmosphere was added a solution of ethyl 3-(4-(sec-butoxy)-2-methylphenyl)-2-methylacrylate (4.65 g, 1 Eq, 16.8 mmol) in anhydrous THF (33.6 mL) and after addition, the mixture was allowed to warm to room temperature and then stirred under reflux for 2 h. The reaction was allowed to cool to room temperature, cooled on ice and dropwise potassium sodium tartrate, saturated solution in water (47.5 g, 112 mL, 1.5 molar, 10 Eq, 168 mmol) was added until gas formation ceased. The solution was extracted using EtOAc, combined organics were washed with water, brine, dried over Na₂SO₄, filtered and concentrated to afford 3-(4-(sec-butoxy)-2-methylphenyl)-2-methylprop-2-en-1-ol (3.73 g, 15 mmol, 88% yield). GCMS: Method 8, 3.17 min, 234.1; calcd. 234.3, ¹H NMR (400 MHz, CDCl₃) δ 7.07 (d, J=8.3 Hz, 1H), 6.76-6.67 (m, 2H), 6.44 (s, 1H), 4.33-4.24 (m, 1H), 4.20 (s, 2H), 2.24 (s, 3H), 1.77 (d, J=1.4 Hz, 3H), 1.74-1.55 (m, 2H), 1.29 (d, J=6.1 Hz, 3H), 0.97 (td, J=7.4, 3.5 Hz, 3H), OH signal not observed.

### Example 39: 4-(sec-butoxy)cyclohexyl)methanol

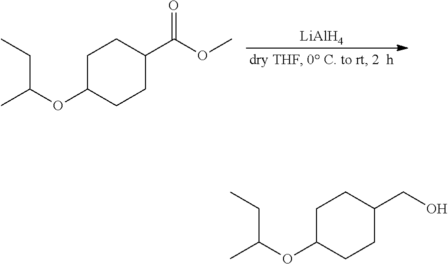

To lithium aluminum hydride (10 mL, 2.4 molar in THF, 3 Eq, 25 mmol) at 0° C. under nitrogen atmosphere was added a solution of methyl 4-(sec-butoxy)cyclohexane-1-carboxylate (1.8 g, 1 Eq, 8.4 mmol) in anhydrous THF (17 mL) and after addition, the mixture was allowed to warm to room temperature and stirred for 2 h. The reaction was cooled on ice and dropwise potassium sodium tartrate, sat. solution in water (56 mL, 1.5 molar, 10 Eq, 84 mmol) was added until gas formation ceased. The solution was extracted using EtOAc, the combined organics were washed with water, brine, dried over Na₂SO₄, filtered and concentrated. Purification by flash column chromatography (SiO₂, 0-100% MTBE in heptane) afforded 4-(sec-butoxy)cyclohexyl)methanol (564.7 mg, 1.516 mmol, 18% yield) (33:67 mixture of isomers). GCMS: Method 8, 2.53 and 2.57 min, 168.0 [fragment mass]; calcd. 186.29, ¹H NMR (400 MHz, CDCl₃) δ 3.53-3.30 (m, 3H), 3.27-3.16 (m, 1H), 2.07-1.95 (m, 1H), 1.88-1.72 (m, 2H), 1.57-1.34 (m, 5H), 1.34-1.15 (m, 2H), 1.15-1.07 (m, 3H), 1.07-0.93 (m, 1H), 0.93-0.81 (m, 3H), OH signal is missing.

### Example 40: 3-(4-(sec-butoxy)-2-methylphenyl)propan-1-ol

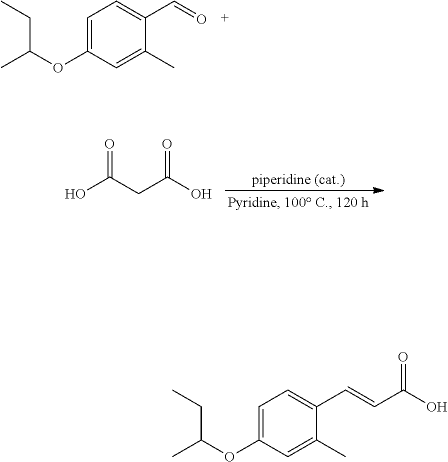 [SMILES: CCC(C)Oc1ccc(C=O)c(C)c1 ; O=C(O)CC(=O)O ; "piperidine (cat.)" ; CCC(C)Oc1ccc(/C=C/C(=O)O)c(C)c1]

### Step 1: (E)-3-(4-(sec-butoxy)-2-methylphenyl)acrylic acid

To 4-(sec-butoxy)-2-methylbenzaldehyde (8.00 g, 1 Eq, 41.6 mmol) in pyridine (100 mL) were added malonic acid (8.66 g, 2 Eq, 83.2 mmol) and piperidine (411 μL, 0.1 Eq, 4.16 mmol) and the mixture was heated at 100° C. for 72 h. Malonic acid (4.33 g, 1 Eq, 41.6 mmol) was added and the mixture was heated at 100° C. for 48 h. The mixture was concentrated, diluted with sat. aq. NaHCO₃ and extracted with DCM. Combined organics were dried over Na₂SO₄, filtered and concentrated. Purification by flash column chromatography (SiO₂, 0-100% [EtOAc/EtOH/AcOH 3:1:0.02 v/v/v] in heptane) afforded (E)-3-(4-(sec-butoxy)-2-methylphenyl)acrylic acid (5.86 g, 18 mmol, 43% yield). ¹H NMR (400 MHz, CDCl₃) δ 10.97 (brs, 1H), 8.01 (d, J=15.8 Hz, 1H), 7.55 (d, J=8.4 Hz, 1H), 6.78-6.66 (m, 2H), 6.29 (d, J=15.8 Hz, 1H), 4.34 (h, J=6.1 Hz, 1H), 2.43 (s, 3H), 1.79-1.57 (m, 2H), 1.30 (d, J=6.1 Hz, 3H), 0.97 (t, J=7.4 Hz, 3H).

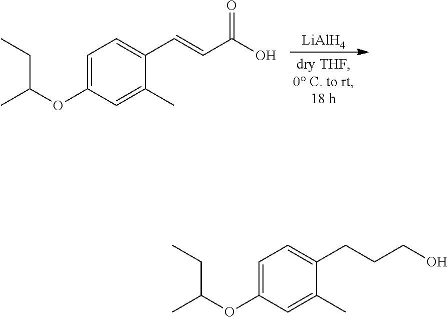 [SMILES: CCC(C)Oc1ccc(/C=C/C(=O)O)c(C)c1 ; "LiAlH4" ; CCC(C)Oc1ccc(CCCO)c(C)c1]

### Step 2: 3-(4-(sec-butoxy)-2-methylphenyl)propan-1-ol

To lithium aluminum hydride (2.27 g, 24.9 mL, 2.4 molar in THF, 4 Eq, 59.7 mmol) at 0° C. under nitrogen atmosphere was added a solution of 3-(4-(sec-butoxy)-2-methylphenyl)acrylic acid (4.86 g, 72% Wt, 1 Eq, 14.9 mmol) in anhydrous THF (29.9 mL) and after addition, the mixture was allowed to warm to room temperature and stirred for 18 h. The reaction was cooled on ice and dropwise potassium sodium tartrate, saturated solution in water (99.6 mL, 1.5 molar, 10 Eq, 149 mmol) was added until gas formation ceased. The mixture was extracted with EtOAc, the combined organics were washed with water, brine, dried over Na₂SO₄, filtered and concentrated. Purification by flash column chromatography (SiO₂, 0-50% MTBE in heptane) afforded 3-(4-(sec-butoxy)-2-methylphenyl)propan-1-ol (0.67 g, 3.05 mmol, 20% yield). GCMS: Method 8, 3.11 min, 222.1; calcd. 222.3, ¹H NMR (400 MHz, CDCl₃) δ 7.03 (d, J=8.3 Hz, 1H), 6.72-6.61 (m, 2H), 4.24 (h, J=5.9 Hz, 1H), 3.70 (td, J=6.3, 5.0 Hz, 2H), 2.71-2.53 (m, 2H), 2.28 (s, 3H), 1.89-1.66 (m, 3H), 1.66-1.57 (m, 1H), 1.27 (d, J=6.1 Hz, 3H), 0.97 (t, J=7.4 Hz, 3H), OH signal is missing.

### Example 41: 3-(4-(sec-butoxy)cyclohexyl)propan-1-ol

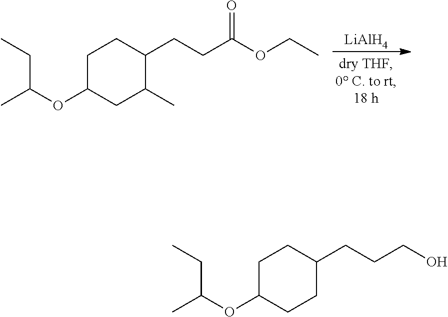

To lithium aluminum hydride (6.53 mL, 2.4 molar in THF, 3 Eq, 15.7 mmol) at 0° C. under nitrogen atmosphere was added a solution of ethyl 3-(4-(sec-butoxy)cyclohexyl)propanoate (1.34 g, 1 Eq, 5.23 mmol) in anhydrous THF (10.5 mL) and after addition, the mixture was allowed to warm to room temperature and stirred for 18 hour. The reaction was cooled on ice and dropwise potassium sodium tartrate solution in water (34.8 mL, 1.5 molar, 10 Eq, 52.3 mmol) was added until gas formation ceased. The solution was extracted using EtOAc, and combined organics were washed with water, brine, dried over Na₂SO₄, filtered and concentrated to afford 3-(4-(sec-butoxy)cyclohexyl)propan-1-ol (0.830 g, 3.87 mmol, 74% yield) (33:67 mixture of isomers). GCMS: Method 8, 2.84 and 2.89 min, 214.0; calcd. 214.3.

### Example 42: 3-(4-(sec-butoxy)-2-methylphenyl)butan-1-ol

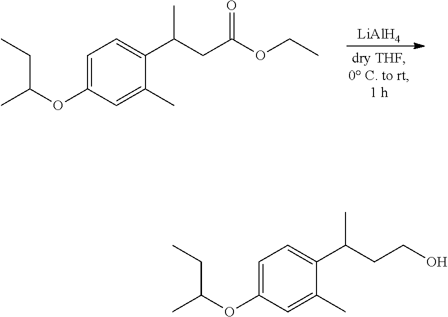

To lithium aluminum hydride (13.5 mL, 2.4 molar in THF, 3 Eq, 32.3 mmol) at 0° C. under nitrogen atmosphere was added a solution of ethyl 3-(4-(sec-butoxy)-2-methylphenyl)butanoate (3.00 g, 1 Eq, 10.8 mmol) in anhydrous THF (21.6 mL). After addition, the mixture was allowed to warm to room temperature and stirred for 1 hour. The reaction was cooled on ice and dropwise potassium sodium tartrate solution in water (71.8 mL, 1.5 molar, 10 Eq, 108 mmol) was added until gas formation ceased. The solution was extracted with EtOAc, and combined organics were washed with water, brine, dried over Na₂SO₄, filtered and concentrated. Purification by flash column chromatography (SiO₂, 0-50% MTBE in heptane) afforded 3-(4-(sec-butoxy)-2-methylphenyl)butan-1-ol (1054.8 mg, 4.4627 mmol, 41% yield). GCMS: Method 8, 3.17 min, 236.1; calcd. 236.3, ¹H NMR (400 MHz, CDCl₃) δ 7.08 (d, J=8.4 Hz, 1H), 6.75-6.66 (m, 2H), 4.24 (h, J=6.1 Hz, 1H), 3.66-3.51 (m, 2H), 3.09 (h, J=7.0 Hz, 1H), 2.30 (s, 3H), 1.92-1.78 (m, 2H), 1.78-1.58 (m, 2H), 1.28 (d, J=6.0 Hz, 3H), 1.21 (d, J=6.9 Hz, 3H), 0.97 (t, J=7.4 Hz, 3H), OH signal is missing.

### Example 43: 3-(4-(sec-butoxy)phenyl)-2-methylbutan-1-ol

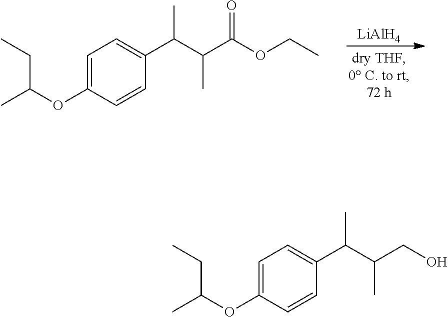

To lithium aluminum hydride (1.85 g, 20.3 mL, 2.4 molar in THF, 3 Eq, 48.8 mmol) at 0° C. under nitrogen atmosphere was added a solution of ethyl 3-(4-(sec-butoxy)phenyl)-2-methylbutanoate (4.53 g, 1 Eq, 16.3 mmol) in anhydrous THF (75 mL) and after addition, the mixture was allowed to warm to 20° C. and stirred at rt for 72 h. The reaction mixture was then cooled down again to 0° C. and potassium sodium tartrate solution in water (45.9 g, 108 mL, 1.5 molar, 10 Eq, 163 mmol) was added dropwise to the mixture. After gas formation ceased, the solution was extracted using EtOAc (3×). The organic fractions were combined, washed with water, brine, dried with Na₂SO₄, filtered and concentrated, affording 3-(4-(sec-butoxy)phenyl)-2-methylbutan-1-ol (3.64 g, 15.4 mmol, 95% yield) as colorless oil (mixture of isomers). GCMS: Method 8, 3.1 min, 236.1; calcd. 236.2. ¹H NMR (400 MHz, CDCl₃) δ 7.14-7.00 (m, 2H), 6.85-6.76 (m, 2H), 4.31-4.08 (m, 1H), 3.65-3.26 (m, 2H), 2.68 (dp, J=53.4, 7.2 Hz, 1H), 1.86-1.58 (m, 3H), 1.30-1.19 (m, 7H), 1.00-0.93 (m, 5H), 0.79 (d, J=6.8 Hz, 1H).

### Example 44: 3-(4-(sec-butoxy)cyclohexyl)-2-methylpropan-1-ol

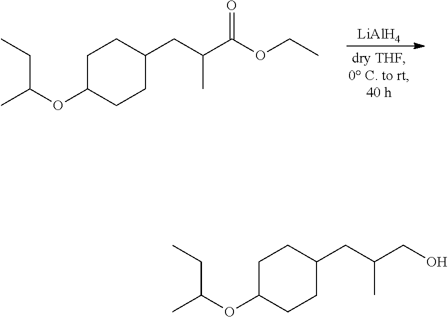

To lithium aluminum hydride (1.24 g, 13.6 mL, 2.4 molar in THF, 3 Eq, 32.6 mmol) at 0° C. under nitrogen atmosphere was added a solution of ethyl 3-(4-(sec-butoxy)cyclohexyl)-2-methylpropanoate (2.94 g, 1 Eq, 10.9 mmol) in anhydrous THF (21.7 mL) and after addition, the mixture was allowed to warm to 20° C. and then stirred at rt for 40 hour. The reaction mixture was then cooled down again to 0° C. and potassium sodium tartrate solution in water (30.7 g, 72.5 mL, 1.5 molar, 10 Eq, 109 mmol) was added dropwise to the mixture. After gas formation ceased, the solution was extracted using EtOAc (3×). The organic fractions were combined, washed with water, brine, dried with Na₂SO₄, filtered and concentrated. The crude was purified by flash column chromatography (SiO₂, 0-50% MBTE in heptane), to give 3-(4-(sec-butoxy)cyclohexyl)-2-methylpropan-1-ol (2.31 g, 10.1 mmol, 93% yield) as a colorless oil (72:28 mixture of isomers). GCMS: Method 8, 2.89 and 2.95 min, 155.1 [main signal-fragment]; calcd. 228.2. ¹H NMR (400 MHz, CDCl₃) δ 3.59-3.44 (m, 1.8H), 3.44-3.30 (m, 1.9H), 3.26-3.13 (m, 0.3H), 2.03-1.86 (m, 0.6H), 1.86-1.64 (m, 3H), 1.57-1.14 (m, 10H), 1.10 (t, J=5.8 Hz, 3H), 1.06-0.72 (m, 8H).

### Example 45: 3-(4-(sec-butoxy)cyclohexyl)butan-1-ol

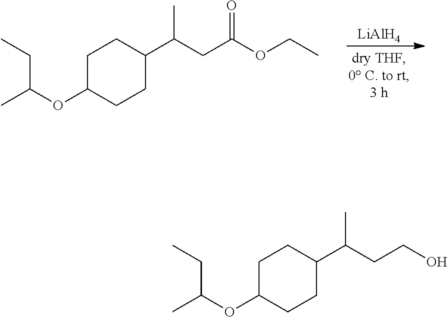

To lithium aluminum hydride (1.28 g, 14.0 mL, 2.4 molar in THF, 3 Eq, 33.6 mmol) at 0° C. under nitrogen atmosphere was added a solution of ethyl 3-(4-(sec-butoxy)cyclohexyl)butanoate (3.03 g, 1 Eq, 11.2 mmol) in anhydrous THF (22.4 mL) and after addition, the mixture was allowed to warm to 20° C. and then stirred at rt for 3 hour. The reaction mixture was then cooled down again to 0° C. and potassium sodium tartrate solution in water (31.6 g, 74.7 mL, 1.5 molar, 10 Eq, 112 mmol) was added dropwise to the mixture. After gas formation ceased, the solution was extracted using EtOAc (3×). The organic fractions were combined, washed with water, brine, dried with Na₂SO₄, filtered and concentrated to give 3-(4-(sec-butoxy)cyclohexyl)butan-1-ol (2.6 g, 11.2 mmol, quant.) as a colorless oil (82:18 mixture of isomers). GCMS: Method 8, 2.89 min and 2.97 min, 155.1 [main signal-fragment]; calcd. 228.2. ¹H NMR (400 MHz, CDCl₃) δ 3.77-3.57 (m, 3H), 3.38-3.32 (m, 1H), 1.83 (dddd, J=15.6, 7.2, 5.7, 2.8 Hz, 2H), 1.74-1.63 (m, 2H), 1.53-1.32 (m, 9H), 1.20 (dt, J=10.7, 5.0 Hz, 2H), 1.10 (t, J=6.0 Hz, 3H), 0.94-0.83 (m, 6H).

### Example 46: 3-(4-(sec-butoxy)-2-methylcyclohexyl)butan-1-ol

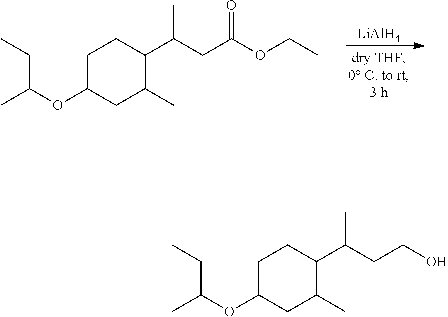

To lithium aluminum hydride (548 mg, 6.02 mL, 2.4 molar in THF, 3 Eq, 14.4 mmol) at 0° C., under nitrogen atmosphere, was added a solution of ethyl 3-(4-(sec-butoxy)-2-methylcyclohexyl)butanoate (1.37 g, 1 Eq, 4.82 mmol) in anhydrous THF (9.63 mL) and after addition, the mixture was allowed to warm to 20° C. and then stirred at rt for 3 h. The reaction mixture was then cooled down again to 0° C. and potassium sodium tartrate solution in water (13.6 g, 32.1 mL, 1.5 molar, 10 Eq, 48.2 mmol) was added dropwise to the mixture. After gas formation ceased, the organics were extracted using EtOAc (3×). The organic layers were combined, washed with water and brine, dried with Na₂SO₄, filtered and concentrated. The crude was purified by flash column chromatography (SiO₂, 0-10% MBTE in heptane then 100% MBTE), affording 3-(4-(sec-butoxy)-2-methylcyclohexyl)butan-1-ol (566.1 mg, 2.335 mmol, 48% yield). GCMS: Method 8, 3.06 min, 95.1 [main signal-fragment]; calcd. 242.403. ¹H NMR (400 MHz, CDCl₃) δ 3.78-3.60 (m, 2H), 3.60-3.50 (m, 1H), 3.42-3.31 (m, 1H), 2.00-1.90 (m, 1H), 1.90-1.65 (m, 3H), 1.56-1.21 (m, 8H), 1.21-1.05 (m, 5H), 1.05-0.97 (m, 3H), 0.97-0.76 (m, 6H).

### Example 47: 3-(4-(sec-butoxy)phenyl)propanal

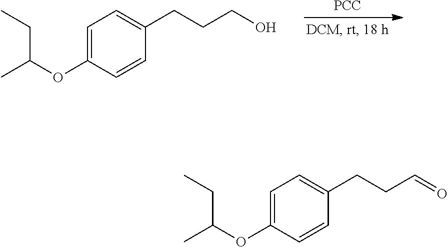

To 3-(4-(sec-butoxy)phenyl)propan-1-ol (4.14 g, 1 Eq, 19.9 mmol) in DCM (300 mL) was added pyridinium chlorochromate (6.43 g, 1.5 Eq, 29.8 mmol) and the mixture was stirred at room temperature for 18 h. The reaction was filtered over celite, the filter cake was rinsed with DCM, and the filtrate was concentrated. Purification by flash column chromatography (SiO₂, 0-50% MTBE in heptane) afforded 3-(4-(sec-butoxy)phenyl)propanal (1.21 g, 5.87 mmol, 29% yield). GCMS: Method 8, 2.88 min, 206.1; calcd. 206.2, ¹H NMR (400 MHz, CDCl₃) δ 9.81 (t, J=1.5 Hz, 1H), 7.17-7.00 (m, 2H), 6.90-6.73 (m, 2H), 4.25 (h, J=6.0 Hz, 1H), 2.90 (t, J=7.5 Hz, 2H), 2.74 (t, J=7.5, 2H), 1.80-1.57 (m, 2H), 1.28 (d, J=6.2 Hz, 3H), 0.97 (t, J=7.4 Hz, 3H).

### Example 48: 3-(4-(sec-butoxy)phenyl)butanal

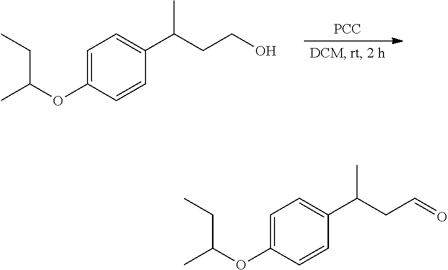 [SMILES: CCC(C)Oc1ccc(C(C)CCO)cc1 ; "PCC" ; CCC(C)Oc1ccc(C(C)CC=O)cc1]

To 3-(4-(sec-butoxy)phenyl)butan-1-ol (590 mg, 1 Eq, 2.65 mmol) in DCM (50 mL) was added pyridinium chlorochromate (858 mg, 1.5 Eq, 3.98 mmol) and the mixture was stirred at room temperature for 2 h. The reaction was filtered over celite, the filter cake was rinsed with DCM, and the filtrate was concentrated. Purification by flash column chromatography (SiO₂, 0-50% MTBE in heptane) afforded 3-(4-(sec-butoxy)phenyl)butanal (245.9 mg, 1.116 mmol, 42% yield). GCMS: Method 8, 2.93 min, 220.1; calcd. 220.3, ¹H NMR (400 MHz, CDCl₃) δ 9.70 (t, J=2.1 Hz, 1H), 7.17-7.05 (m, 2H), 6.86-6.79 (m, 2H), 4.25 (h, J=6.1 Hz, 1H), 3.31 (h, J=7.1 Hz, 1H), 2.76-2.51 (m, 2H), 1.80-1.58 (m, 2H), 1.29-1.27 (m, 6H), 0.97 (t, J=7.5 Hz, 3H).

### Example 49: 3-(4-(sec-butoxy)-2-methylphenyl)-2-

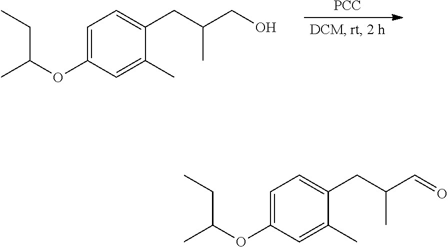 [SMILES: CCC(C)Oc1ccc(CC(C)CO)c(C)c1 ; "PCC" ; CCC(C)Oc1ccc(CC(C)C=O)c(C)c1]

To 3-(4-(sec-butoxy)-2-methylphenyl)-2-methylpropan-1-ol (1.39 g, 1 Eq, 5.88 mmol; prepared in Example 28) in DCM (118 mL) was added pyridinium chlorochromate (1.90 g, 1.5 Eq, 8.82 mmol) and mixture stirred at room temperature for 2 hours. The mixture was filtered over celite, the filter cake was rinsed with DCM, and the filtrate was concentrated. Purification by flash column chromatography (SiO₂, 0-50% MTBE in heptane) afforded 3-(4-(sec-butoxy)-2-methylphenyl)-2-methylpropanal (950.5 mg, 4.056 mmol, 69% yield). GCMS: Method 8, 3.05 min, 234.1; calcd. 234.3, ¹H NMR (400 MHz, CDCl₃) δ 9.71 (d, J=1.6 Hz, 1H), 6.99 (d, J=8.3 Hz, 1H), 6.71 (d, J=2.7 Hz, 1H), 6.66 (dd, J=8.3, 2.7 Hz, 1H), 4.25 (h, J=6.1 Hz, 1H), 3.02 (dd, J=14.0, 6.2 Hz, 1H), 2.68-2.55 (m, 1H), 2.50 (dd, J=14.0, 8.3 Hz, 1H), 2.27 (s, 3H), 1.82-1.53 (m, 2H), 1.27 (d, J=6.1 Hz, 3H), 1.10 (d, J=7.0 Hz, 3H), 0.97 (t, J=7.4 Hz, 3H).

### Example 50: 3-(4-(sec-butoxy)phenyl)-2-methylpropanal

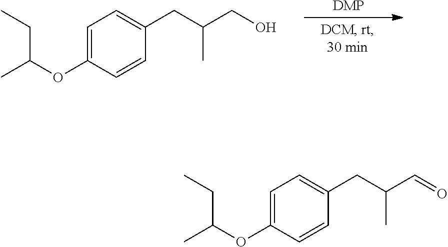

To 3-(4-(sec-butoxy)phenyl)-2-methylpropan-1-ol (1.17 g, 1 Eq, 5.26 mmol) in dichloromethane (26.3 mL) was added Dess-Martin periodinane (2.46 g, 1.1 Eq, 5.79 mmol) and water (104 L, 1.1 Eq, 5.79 mmol) dropwise. The mixture was stirred at room temperature for 30 min, diluted with sat. aq. NaHCO₃ and water and layers separated. The organic phase was washed with brine, dried over Na₂SO₄, filtered and concentrated. Purification by flash column chromatography (SiO₂, 0-50% MTBE in heptane) afforded 3-(4-(sec-butoxy)phenyl)-2-methylpropanal (624.5 mg, 2.835 mmol, 54% yield). GCMS: Method 8, 2.95 min, 220.1; calcd. 220.3, ¹H NMR (400 MHz, CDCl₃) δ 9.71 (d, J=1.6 Hz, 1H), 7.10-7.00 (m, 2H), 6.87-6.75 (m, 2H), 4.25 (h, J=6.1 Hz, 1H), 3.01 (dd, J=13.5, 5.8 Hz, 1H), 2.69-2.58 (m, 1H), 2.55 (dd, J=13.5, 8.1 Hz, 1H), 1.80-1.67 (m, 1H), 1.67-1.58 (m, 1H), 1.28 (d, J=6.0 Hz, 3H), 1.08 (d, J=6.8 Hz, 3H), 0.97 (t, J=7.5 Hz, 3H).

### Example 51: 3-(4-(sec-butoxy)-2-methylphenyl)propanal

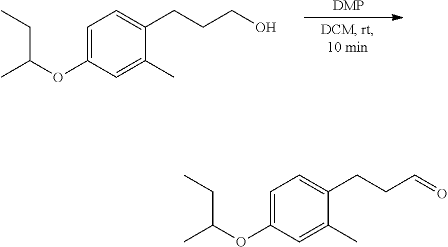

To 3-(4-(sec-butoxy)-2-methylphenyl)propan-1-ol (0.44 g, 1 Eq, 2.0 mmol) and Dess-Martin periodinane (1.0 g, 1.2 Eq, 2.4 mmol) in dichloromethane (9.9 mL) was added dropwise water (39 μL, 1.1 Eq, 2.2 mmol) and the mixture was stirred at room temperature for 30 min. The mixture was quenched by addition of ethanol and concentrated. Purification by flash column chromatography (SiO₂, 0-50% MTBE in heptane) afforded 3-(4-(sec-butoxy)-2-methylphenyl)propanal (273.8 mg, 1.243 mmol, 63% yield). GCMS: Method 8, 3.02 min, 220.1; calcd. 220.3, ¹H NMR (400 MHz, CDCl₃) δ 9.84 (t, J=1.5 Hz, 1H), 7.01 (d, J=8.3 Hz, 1H), 6.74-6.63 (m, 2H), 4.24 (h, J=6.1 Hz, 1H), 2.95-2.83 (m, 2H), 2.74-2.65 (m, 2H), 2.27 (s, 3H), 1.79-1.56 (m, 2H), 1.27 (d, J=6.1 Hz, 3H), 0.97 (t, J=7.5 Hz, 3H).

### Example 52: 4-(sec-butoxy)cyclohexane-1-carbaldehyde

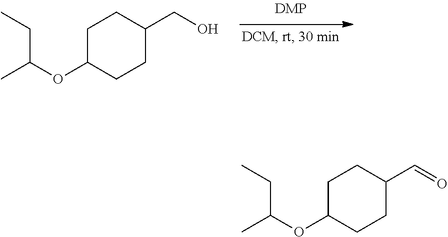

To (4-(sec-butoxy)cyclohexyl)methanol (2.0 g, 1 Eq, 11 mmol) and Dess-Martin periodinane (5.5 g, 1.2 Eq, 13 mmol) in dichloromethane (54 mL) was added dropwise water (0.21 mL, 1.1 Eq, 12 mmol) and the mixture was stirred at room temperature for 30 min. The mixture was quenched by addition of ethanol, and concentrated. Purification by flash column chromatography (SiO₂, 0-50% MTBE in heptane) afforded 4-(sec-butoxy)cyclohexane-1-carbaldehyde (1.35 g, 7.33 mmol, 68% yield). GCMS: Method 8, 2.48 min, 184.1; calcd. 184.2.

### Example 53: 3-(4-(sec-butoxy)cyclohexyl)propanal

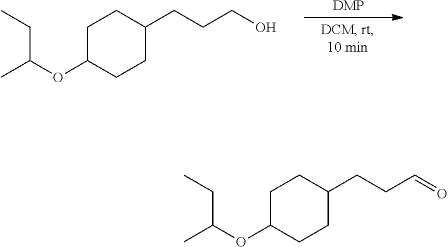

To 3-(4-(sec-butoxy)cyclohexyl)propan-1-ol (0.830 g, 1 Eq, 3.87 mmol) and Dess-Martin periodinane (1.97 g, 1.2 Eq, 4.65 mmol) in dichloromethane (19.4 mL) was added dropwise water (76.8 L, 1.1 Eq, 4.26 mmol) and the mixture was stirred at room temperature for 10 min. The mixture was quenched by addition of ethanol, and concentrated. Purification by flash column chromatography (SiO₂, 0-60% MTBE in heptane) afforded 3-(4-(sec-butoxy)cyclohexyl)propanal (483.3 mg, 2.276 mmol, 59% yield) (33:67 mixture of isomers). GCMS: Method 8, 2.73 and 2.78 min, 183.1 [fragment mass]; calcd. 212.3. ¹H NMR (400 MHz, CDCl₃) δ 9.79-9.72 (m, 1H), 3.59-3.52 (m, 0.4H), 3.46-3.38 (m, 0.6H), 3.38-3.30 (m, 0.5H), 3.25-3.15 (m, 0.5H), 2.48-2.38 (m, 2H), 2.03-1.91 (m, 1H), 1.83-1.70 (m, 2H), 1.61-1.54 (m, 2H), 1.53-1.30 (m, 5H), 1.30-1.12 (m, 2H), 1.10 (t, J=5.7 Hz, 3H), 1.01-0.84 (m, 4H).

### Example 54: 3-(4-(sec-butoxy)-2-methylphenyl)butanal

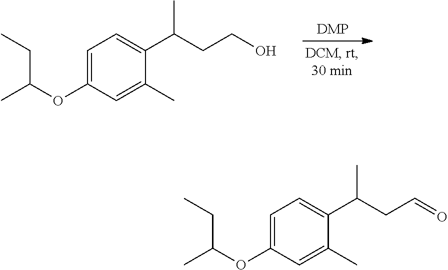

To 3-(4-(sec-butoxy)-2-methylphenyl)butan-1-ol (1.40 g, 1 Eq, 5.92 mmol) and Dess-Martin periodinane (3.01 g, 1.2 Eq, 7.11 mmol) in dichloromethane (29.6 mL) was added dropwise water (117 L, 1.1 Eq, 6.52 mmol) and the mixture was stirred at room temperature for 30 min. The mixture was quenched by addition of ethanol, and concentrated. Purification by flash column chromatography (SiO₂, 0-60% MTBE in heptane) afforded 3-(4-(sec-butoxy)-2-methylphenyl)butanal (1079.4 mg, 4.3 mmol, 72% yield). GCMS: Method 8, 3.04 min, 234.1; calcd. 234.3, ¹H NMR (400 MHz, CDCl₃) δ 9.70 (t, J=2.0 Hz, 1H), 7.06 (d, J=8.1 Hz, 1H), 6.74-6.65 (m, 2H), 4.25 (h, J=6.0 Hz, 1H), 3.58-3.44 (m, 1H), 2.76-2.50 (m, 2H), 2.33 (s, 3H), 1.80-1.66 (m, 1H), 1.66-1.57 (m, 1H), 1.27 (d, J=6.1 Hz, 3H), 1.25 (d, J=6.9 Hz, 3H), 0.97 (t, J=7.5 Hz, 3H).

### Example 55: 3-(4-(sec-butoxy)phenyl)-2-methylbutanal

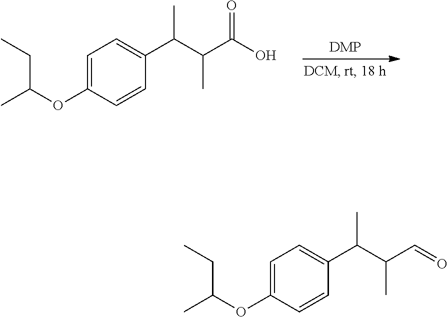

To a solution of 3-(4-(sec-butoxy)phenyl)-2-methylbutan-1-ol (1.50 g, 1 Eq, 6.35 mmol) in dichloromethane (60 mL) was added Dess-Martin periodinane (3.23 g, 2.36 mL, 1.2 Eq, 7.62 mmol) and the resulting mixture was stirred at rt for 18 h. The reaction was stopped and then quenched with sat. Na₂SO₃, diluted with diethyl ether and phases were separated. The aqueous layer was extracted once more with diethyl ether and combined organics were washed with sat. NaHCO₃, brine, dried over Na₂SO₄, filtered and concentrated. The crude was purified by flash column chromatography (SiO₂, 0-100% MBTE in heptane), affording 3-(4-(sec-butoxy)phenyl)-2-methylbutanal (1290.6 mg, 5.5074 mmol, 87% yield) as a colorless oil (58:42 mixture of isomers). GCMS: Method 8, 3.0 and 3.03 min, 234.1; calcd. 234.2. ¹H NMR (400 MHz, CDCl₃) δ 9.63 (dd, J=35.7, 2.6 Hz, 1H), 7.13-7.01 (m, 2H), 6.87-6.78 (m, 2H), 4.26 (hd, J=6.1, 2.5 Hz, 1H), 3.14-2.89 (m, 1H), 2.62-2.44 (m, 1H), 1.81-1.57 (m, 2H), 1.30-1.24 (m, 6H), 1.07 (d, J=7.0 Hz, 2H), 0.97 (td, J=7.4, 1.9 Hz, 3H), 0.90 (d, J=6.9 Hz, 1H).

### Example 56: 3-(4-(sec-butoxy)cyclohexyl)butanal

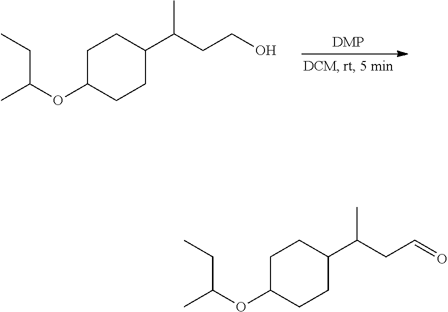

3-(4-(sec-butoxy)cyclohexyl)butan-1-ol (1.0 g, 1 Eq, 4.4 mmol) and Dess-Martin periodinane (2.2 g, 1.2 Eq, 5.3 mmol) were suspended in dichloromethane (22 mL) and then water (87 mg, 87 μL, 1.1 Eq, 4.8 mmol) was added dropwise. The suspension was stirred at rt for few minutes until reaching completion. The reaction was stopped, and the mixture was quenched with sat. K₂SO₃ solution, diluted with diethyl ether, and the phases were separated. The organic layer was washed first with sat. NaHCO₃ solution, then with brine, Na₂SO₄, filtered and concentrated. The crude was purified by flash column chromatography (SiO₂, 0-50% MBTE in heptane) to give 3-(4-(sec-butoxy)cyclohexyl)butanal (258.8 mg, 1.143 mmol, 26% yield) as a colorless oil. GCMS: Method 8, 2.81 min, 135.1 [main signal-fragment]; calcd. 226.2. ¹H NMR (400 MHz, CDCl3) δ 9.76 (dd, J=3.0, 1.8 Hz, 1H), 3.64-3.57 (m, 1H), 3.34 (h, J=6.1 Hz, 1H), 2.53-2.43 (m, 1H), 2.25-2.14 (m, 1H), 2.06-1.93 (m, 1H), 1.91-1.77 (m, 2H), 1.56-1.15 (m, 10H), 1.09 (d, J=6.1 Hz, 3H), 0.96-0.89 (m, 6H).

### Example 62: Odor Profile Determination

Odor profile for selected compounds within the scope of the disclosure is determined using assessments by one Master Perfumer and 4-6 trained sensory panelists. Sensory panelists are trained on our internal taxonomy with reference materials that include 11 grand families, and 62 subfamilies. Panelists are experienced in, but not trained in, an additional several hundred odor descriptors. Panelists are trained on usage of a rating scale, which also includes internal references. Panelists are not advanced to compound evaluation until they have completed and received a passing score on a Final Exam on the taxonomy that was curated by our perfumery team.

A single score is generated for the sensory panel by taking the arithmetic mean of all panelist scores for a given sample, for a given attribute. Alternatively, a single score may be generated by fitting all panelist scores to a model that can correct for differences in the intercept or slope of the latent function through which each panelist produces a rating given a percept, i.e. correction for inter-panelist variation.

The Master Perfumer, possessing a deep knowledge of fragrance ingredients, high level technical expertise, and who is recognized for their experience in creating complex perfumes, reviews, may augment sensory panel scores for improved accuracy.

Test samples are dissolved at a concentration of 10% w/v in ethanol. At time 0 (t 0), new test blotters (White Paper Paddle Shaped Perfumery Blotters, measuring 5×0.5 inches) are dipped into the 10% solution of the text compound. Odor descriptions at time t=0 are captured within 1-2 minutes of wetting the blotter to allow for evaporation of most of the ethanol solvent, permitting a more accurate determination of the test compound's odor. Assessments are made at ambient temperature in a benchtop laboratory setting. The procedure is repeated at 2 hours and at 24 hours.

The results are shown in the table below:

Intensity: 1 - 10 (max) at t = 0, 10% EtOH solution on blotter paper
+ = 0-3; ++ = 4-7; +++ = 8-10

| | | | Intensity | Intensity | Intensity |
|---|---|---|---|---|---|
| Ex. | | | (1-10) at | (1-10) at | (1-10) at |
| No. | Structure | Primary Notes | 0 hours | 2 hours | 24 hours |
| 2 | 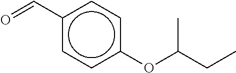 | floral, citrus, violet, orris, ginger, lemongrass | ++ | ++ | ++ |
| 55 | 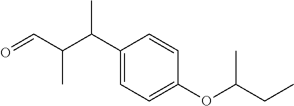 [SMILES: CCC(C)Oc1ccc(C(C)C(C)C=O)cc1] | green | + | ++ | + |
| 43 | 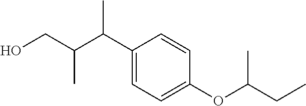 [SMILES: CCC(C)Oc1ccc(C(C)C(C)CO)cc1] | weak | + | + | + |
| 45 | 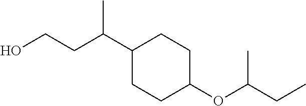 [SMILES: CCC(C)OC1CCC(C(C)CCO)CC1] | green, floral, mineral | +++ | ++ | ++ |
| 56 | 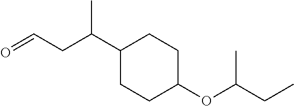 [SMILES: CCC(C)OC1CCC(C(C)CC=O)CC1] | floral, green | ++ | + | + |
| 46 | 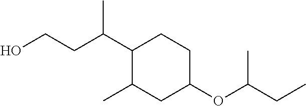 [SMILES: CCC(C)OC1CCC(C(C)CCO)C(C)C1] | woody, gourmand, leather, saffron | + | + | + |
| 8 | 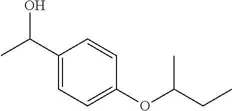 | herbal, lavender, metallic, fruity, mossy | ++ | ++ | ++ |
| 30 | 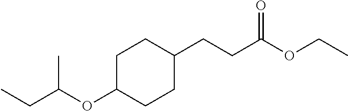 [SMILES: CCOC(=O)CCC1CCC(OC(C)CC)CC1] | green, floral, woody | ++ | + | + |
| 53 | 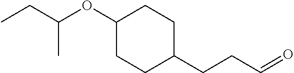 | herbal, woody, medicinal, cooling | +++ | + | + |
| 31 | 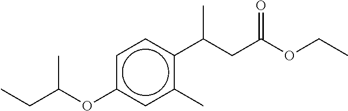 | woody, citrus | ++ | + | + |
| 42 | 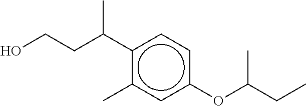 | fruity, floral, woody, green | ++ | + | + |
| 54 | 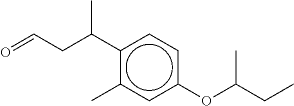 | mineral, aldehydic, weak | + | ++ | ++ |
| 49 | 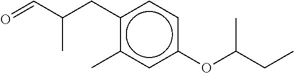 | muguet, fresh | ++ | ++ | ++ |
| 28 | 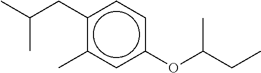 | floral, rosy, mineral, industrial, fruity | ++ | + | + |
| 29 | 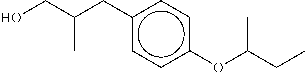 | fruity, sweet balsamic, mineral, woody, floral | ++ | + | + |
| 39 | 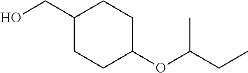 | weak | + | + | + |
| 50 | 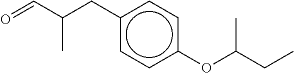 | powdery, green, spicy | ++ | ++ | + |
| 40 | 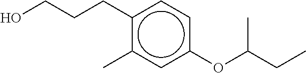 | fruity, rosy, roasted | + | + | + |
| 51 | 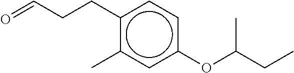 | muguet, fresh, fruity, elegant | ++ | ++ | ++ |
| 12 | 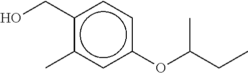 | coconut, floral, weak | ++ | + | + |
| 10 | 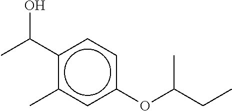 | weak | + | + | + |
| 19 | 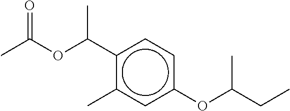 | woody, ozonic, pine | ++ | + | + |
| 20 | 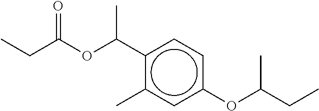 | fruity, wet, industrial, inky | ++ | + | + |
| 6 | 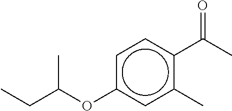 | floral, white floral, starchy, muguet | ++ | ++ | + |
| 7 | 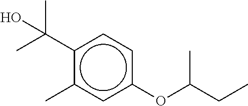 | weak | + | + | + |
| 37 | 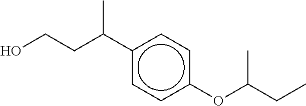 | sweet, floral | ++ | + | + |
| 36 | 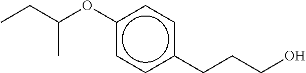 | weak | + | + | + |
| 48 | 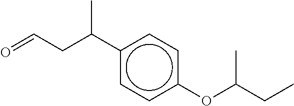 | floral, waxy, fatty | +++ | ++ | + |
| 1 | 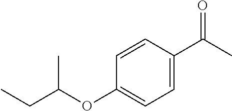 | soulful, wet, anisic, ricey | ++ | ++ | ++ |
| 13 | 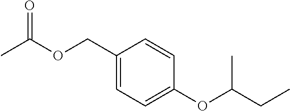 [SMILES: CCC(C)Oc1ccc(COC(C)=O)cc1] | floral, lactonic, rosy | ++ | + | + |
| 14 | 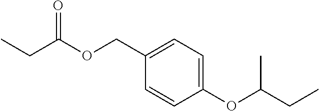 [SMILES: CCC(=O)OCc1ccc(OC(C)CC)cc1] | floral, lactonic, green | ++ | + | + |
| 57 | 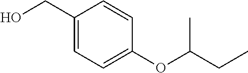 | fruity, gourmand, rosy | ++ | + | + |
| 15 | 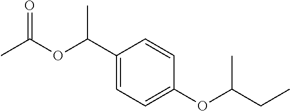 [SMILES: CCC(C)Oc1ccc(C(C)OC(C)=O)cc1] | weak | + | + | + |
| 16 | 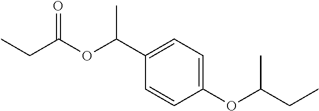 [SMILES: CCC(=O)OC(C)c1ccc(OC(C)CC)cc1] | sweet, lactonic | + | + | + |
| 3 | 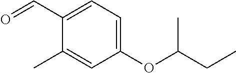 | starchy, floral, ambery, slightly cuminic | ++ | ++ | + |
| 4 | 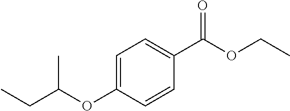 [SMILES: CCOC(=O)c1ccc(OC(C)CC)cc1] | floral, lactonic | + | + | + |
| 18 | 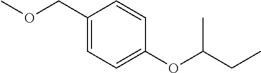 | green, muguet, aldehydic | ++ | ++ | + |
| 17 | 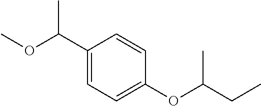 | soulful, driftwood, lactonic, white floral | ++ | ++ | ++ |
| 9 | 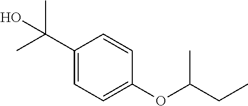 | woody, slightly fruity | + | + | + |
| 58 | 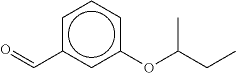 | floral, rosy, powdery, herbal, metallic, marine | ++ | ++ | ++ |
| 59 | 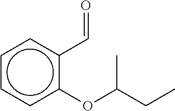 | green, wet, starchy, aldehydic | ++ | ++ | ++ |
| 47 | 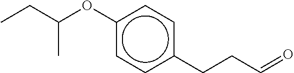 | nutty, floral, green | ++ | ++ | + |
| 44 | 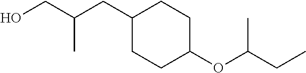 [SMILES: CCC(C)OC1CCC(CC(C)CO)CC1] | phenolic | + | + | + |
| 5 | 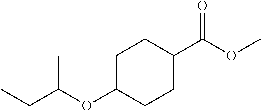 | TBD | | | |
| 11 | 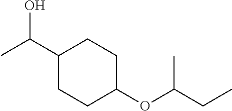 | TBD | | | |
| 21 | 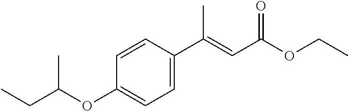 [SMILES: CCOC(=O)/C=C(\C)c1ccc(OC(C)CC)cc1] | TBD | | | |
| 22 | 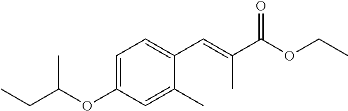 [SMILES: CCOC(=O)/C(C)=C/c1ccc(OC(C)CC)cc1C] | TBD | | | |
| 23 | 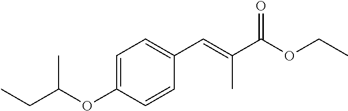 [SMILES: CCOC(=O)/C(C)=C/c1ccc(OC(C)CC)cc1] | TBD | | | |
| 24 | 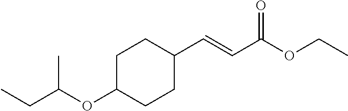 [SMILES: CCOC(=O)/C=C/C1CCC(OC(C)CC)CC1] | TBD | | | |
| 25 | 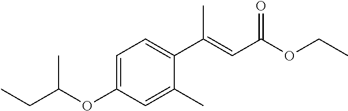 [SMILES: CCOC(=O)/C=C(\C)c1ccc(OC(C)CC)cc1C] | TBD | | | |
| 26 | 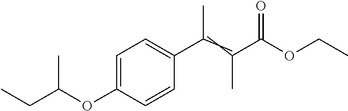 [SMILES: CCOC(=O)C(C)=C(C)c1ccc(OC(C)CC)cc1] | TBD | | | |
| 27 | 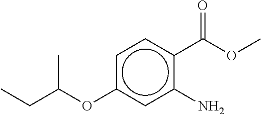 | grapefruit, apple, red fruit | ++ | ++ | ++ |
| 32 | 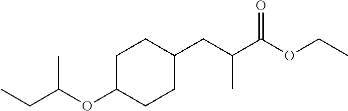 [SMILES: CCOC(=O)C(C)CC1CCC(OC(C)CC)CC1] | TBD | | | |
| 33 | 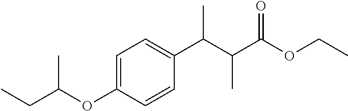 [SMILES: CCOC(=O)C(C)C(C)c1ccc(OC(C)CC)cc1] | TBD | | | |
| 34 | 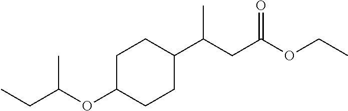 [SMILES: CCOC(=O)CC(C)C1CCC(OC(C)CC)CC1] | TBD | | | |
| 35 | 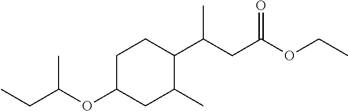 [SMILES: CCOC(=O)CC(C)C1CCC(OC(C)CC)CC1C] | TBD | | | |
| 38 | 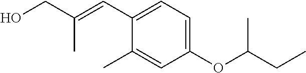 [SMILES: CCC(C)Oc1ccc(/C=C(\C)CO)c(C)c1] | TBD | | | |
| 41 | 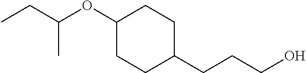 | TBD | | | |
| 52 | 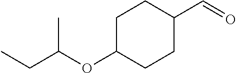 | TBD | | | |
| 60 | 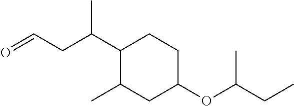 [SMILES: CCC(C)OC1CCC(C(C)CC=O)C(C)C1] | TBD | | | |
| 61 | 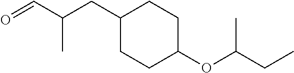 [SMILES: CCC(C)OC1CCC(CC(C)C=O)CC1] | TBD | | | |

The Examples provided herein are exemplary only and are not intended to be limiting in any way to the various aspects and embodiments of the invention described herein.

1. A compound of Formula A: 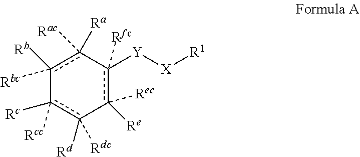
wherein:
Y is C₁₋₆alkylene or C₂₋₆alkenylene, or Y is absent;
X is —O—, —C(O)—, —OC(O)—, or —C(O))—, or X is absent;
one of R^{a}, R^{b}, R_{c}, R^{d}, and R^{e}, is OR², and the remaining four of R^{a}, R^{b}, R^{c}, R^{d}, and R^{e}, is independently a group R;
each of R^{ac}, R^{bc}, R^{cc}, R^{dc}, R^{ec}, and R^{fc}, if present, is independently selected from H, C₁₋₃alkyl (e.g., methyl), and OH;
R¹ is H, OH, optionally substituted C₁₋₆alkyl, optionally substituted C₃₋₆cycloalkyl, or OR³;
provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is each independently selected from H, C₁₋₃alkyl (e.g., methyl), OH, and NH₂.

2. The compound of Formula A according to claim 1, wherein the compound is a compound of Formula I: 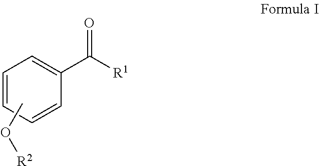
wherein:
R¹ is H, OH, optionally substituted C₁₋₆alkyl, optionally substituted C₃₋₆cycloalkyl or OR³;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl,
optionally provided that the compound is not 4-sec-butoxy-1-benzaldehyde.

3. The compound of Formula A according to claim 1, wherein the compound is a compound of Formula II: 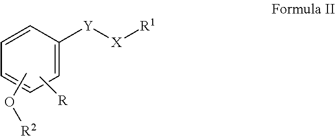
wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³;
provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂;
optionally provided that the compound is not 4-sec-butoxy-1-benzaldehyde.

4. The compound of Formula A according to claim 1, wherein the compound is a compound of Formula III: 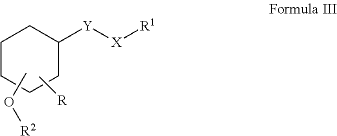
wherein:
Y is C₁₋₆alkylene or C₂₋₆alkenylene, or Y is absent;
X is —O—, —C(O)—, —OC(O)—, or —C(O))—, or X is absent;
R¹ is H, OH, optionally substituted C₁₋₆alkyl, optionally substituted C₃₋₆cycloalkyl, or OR³;
provided that R¹ is not OH when X is —O—;
R² is optionally substituted C₁₋₁₃alkyl (e.g., C₁₋₅alkyl, C₁₋₇alkyl, C₁₋₉alkyl);
R³ is C₁₋₆alkyl or C₃₋₆cycloalkyl; and
R is H, C₁₋₃alkyl (e.g., methyl), OH, or NH₂.

5. (canceled)

6. (canceled)

7. (canceled)

8. A flavor composition and/or fragrance composition comprising a compound of Formula A, according to claim 1, in admixture with one or more non-toxic, orally acceptable, pharmaceutically acceptable, cosmetically acceptable, or acceptable for a household product, carriers or excipients.

9. A flavor composition and/or fragrance composition comprising a compound of Formula I, according to claim 2, in admixture with one or more non-toxic, orally acceptable, pharmaceutically acceptable, cosmetically acceptable, or acceptable for a household product, carriers or excipients.

10. A flavor composition and/or fragrance composition comprising a compound of Formula II, according to claim 3, in admixture with one or more non-toxic, orally acceptable, pharmaceutically acceptable, cosmetically acceptable, or acceptable for a household product, carriers or excipients.

11. A flavor composition and/or fragrance composition comprising a compound of Formula III, according to claim 4, in admixture with one or more non-toxic, orally acceptable, pharmaceutically acceptable, cosmetically acceptable, or acceptable for a household product, carriers or excipients.

12. The composition of claim 9, wherein the compound of Formula I is 4-sec-butoxy-1-benzaldehyde.

13. The composition of claim 10, wherein the compound of Formula II is: 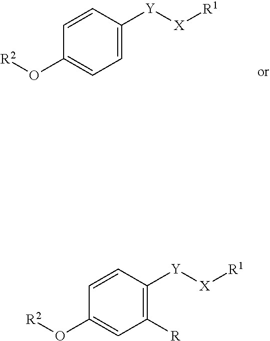
wherein Y, X, R, R¹ and R² are as defined in claim 10.

14. The composition of claim 10, wherein the compound of Formula II is: 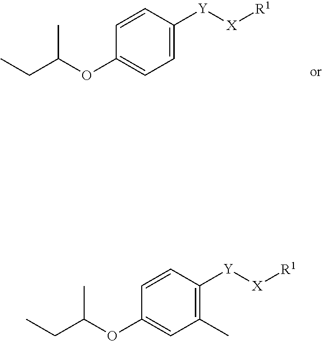
wherein Y, X, and R¹ are as defined in claim 10.

15. The composition of claim 10, wherein the compound of Formula II is selected from any one or more of the compounds of Examples 1-4, 6-10, 12-23, 25-29, 31, 33, 36-38, 40, 42, 43, 47-51, 54, 55, and 57-59, as defined herein.

16. The composition of claim 11, wherein the compound of Formula III is: 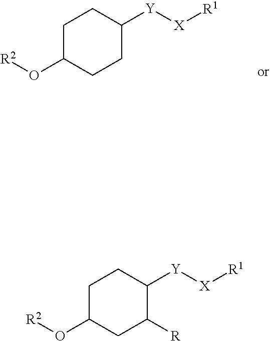
wherein Y, X, R, R¹ and R² are as defined in claim 11.

17. The composition of claim 11, wherein the compound of Formula III is: 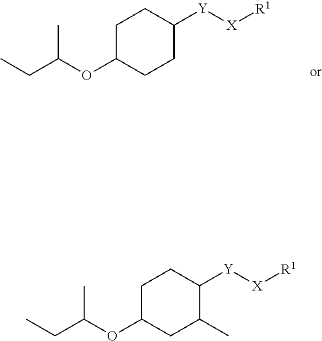
wherein Y, X, and R¹ are as defined in claim 11.

18. The composition of claim 11, wherein the compound of Formula III is selected from any one or more of the compounds of Examples 5, 11, 24, 30, 32, 34, 35, 39, 41, 44, 45, 46, 52, 53, 56, 60, or 61, as defined herein.

19. The composition of claim 8, wherein the composition further comprises one or more solvents and/or one or more other flavors or fragrances.

20. A product comprising the composition of claim 8, e.g., a product selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.
